(19) United States
(12) Patent Application Publication
(10) Pub. No.: US 2023/0293322 A1
(43) Pub. Date: Sep. 21, 2023

(54) FLEXIBLE MULTI USE POST OPERATIVE PROSTHETIC SOCKET SYSTEM

(71) Applicant: Robert N Brown, Geneva, NY (US)

(72) Inventor: Robert N Brown, Geneva, NY (US)

(21) Appl. No.: 18/128,519

(22) Filed: Mar. 30, 2023

Related U.S. Application Data
(63) Continuation-in-part of application No. 17/449,155, filed on Sep. 28, 2021.
Application No. 17/449,155 is a division of application No. 16/486,301, filed on Aug. 15, 2019, now Pat. No. 11,304,830.
(60) Provisional application No. 62/696,043, filed on Jul. 10, 2018.
(60) Provisional application No. 62/667,817, filed on May 7, 2018.
(60) Provisional application No. 62/636,542, filed on Feb. 28, 2018.
(60) Provisional application No. 62/570,731, filed on Oct. 11, 2017.

(51) Int. Cl. A61F 2/80; A61F 2/78

(52) U.S. Cl. CPC A61F 2/80; A61F 2/7812; A61F 2002/5007

(57) ABSTRACT

A single socket adjustable prosthetic system having a first shell with an extended surface configured to fit against a first side of an amputee's limb, the surface terminating at a base; a second shell configured to fit against a second side of the amputee's limb; an articulated hinge having a first member, a middle member, and a second member, the first member having a first plate connected to a second plate at an angle to each other, with the second plate hingedly connected to a first end of the middle member and the second member hingedly attached at a second end of the middle member, the first plate removably connected to the base and the second member of the articulated hinge attached to the second shell; a space between the first shell and the second shell, to be varied to hold, and accommodate the amputee's limb.

### CROSS-REFERENCE TO RELATED APPLICATIONS

This application is a Continuation in Part of copending application Ser. No. 17/449,155 entitled A FLEXIBLE MULTI USE POST OPERATIVE PROSTHETIC SOCKET SYSTEM, filed on Sep. 28, 2021, which is a divisional of application Ser. No. 16/486,301, entitled A FLEXIBLE MULTI USE POST OPERATIVE PROSTHETIC SOCKET SYSTEM which is a U.S. National Stage Application filed on Aug. 15, 2019, which claims the benefit of and priority under 35 USC 371 of International Patent Application PCT/US2018/055155 entitled A FLEXIBLE MULTI USE POST OPERATIVE PROSTHETIC SOCKET SYSTEM, filed on Oct. 10, 2018 which claims the benefit of and priority under the applicable laws of the United States of: U.S. Provisional Application Ser. No. 62/696,043 filed on Jul. 10, 2018 entitled A FLEXIBLE MULTI USE POST OPERATIVE PROSTHETIC SOCKET SYSTEM; U.S. Provisional Application Ser. No. 62/667,817 filed on May 7, 2018 entitled A FLEXIBLE MULTI USE POST OPERATIVE PROSTHETIC SOCKET SYSTEM; U.S. Provisional Application Ser. No. 62/636,542 filed on Feb. 28, 2018 entitled A FLEXIBLE MULTI USE POST OPERATIVE PROSTHETIC SOCKET SYSTEM; and U.S. Provisional Application Ser. No. 62/570,731 filed on Oct. 11, 2017 entitled A FLEXIBLE MULTI USE POST OPERATIVE PROSTHETIC SOCKET SYSTEM the contents of all four of which are relied upon and incorporated herein by reference in its entirety.

### BACKGROUND OF THE INVENTION

### Technical Field

The disclosure relates generally to a system and apparatus for post-operative prosthetic devices for leg amputees and more particularly for trans-tibial amputees.

### Background Information

The present invention is an improvement over the inventions described in U.S. Pat. No. 5,571,209 for a “Post-Operative Protective Prosthesis” (the '209 patent) and U.S. Pat. No. 5,728,165 for an “Adjustable Post-Operative Prosthetic System,” (the '165 patent). Both patents—the '209 patent and the '165 patent—are incorporated herein by reference as if set forth herein at length. The '209 discloses a post-operative adjustable protective socket for a patient that has undergone a trans-tibial amputation. The '165 patent added an outer socket 110 (FIG. 3 of the '165 patent), a pylon 126, and prosthetic foot. Thus, the post-operative protective socket 10 could be inserted into the outer socket with pylon and prosthetic foot attached to allow the amputee to stand and walk.

The post-operative preparatory socket 10 is designed with a rear shell 13. As stated in the '165 patent, “The rear shell 13 is brought to a higher elevation so it passes over the wearer's knee and covers a portion of the wearer's thigh above the knee.” (Lines 2 to 4 of the '165 patent). Preparatory Socket 10 was designed to immobilize the knee during the initial recovery of the amputee. Movement of the knee after the operation before it has sufficient time to heat could result in a reopening of the wound and other complications.

The '165 patent further states: After additional healing has occurred and the amputation is ready for full weight bearing, the patient will no longer need the post-operative preparatory socket 10. Instead, a supra patellar socket 210 is indicated (FIG. 2). (Column 5, lines 37 to 40) The supra patellar socket 210 was designed to allow the knee to flex for full mobility when inserted into outer socket 110 with pylon 126 and prosthetic foot 130.

Although unique and very functional, the system described by the '209 and '165 patents still required three different sockets. Additionally, to accommodate a wide spectrum of limb sizes of amputees at least 5 different sizes of post-operative protective sockets 10 and supra patellar sockets 210 were needed. This is due to the limitation of adjustability of the sockets 10 and 210.

No admission is made that any reference cited herein constitutes prior art. Applicant expressly reserves the right to challenge the accuracy and pertinence of any cited documents.

### SUMMARY OF THE INVENTION

Among the objectives of the present invention is to provide an adjustable prosthetic system for an amputee that can be adjusted for changes in the size and condition of an amputee's limb during post-operative recovery and rehabilitation.

It is an objective of the present invention to provide an interface socket that can initially restrict movement of the leg of the amputee during post-operative recovery and then can be modified to allow movement of the amputee's knee joint.

It is an objective of the present invention to reduce the number of sockets needed to accommodate the varying leg sizes of amputees.

It is an objective of the present invention to provide a prosthetic system that can be modified and adjusted to meet the changing needs of a post-operative amputee during the recovery and physical therapy stages without the need to provide more than one set of sockets.

In one aspect of the present disclosure provided herein, is a single socket adjustable prosthetic system having a first shell with an extended surface configured to fit against a first side of an amputee's limb, the surface terminating at a base, the base configured to extend under the amputee's limb and the base having a limb facing surface and a bottom surface; a second shell with an extended surface configured to fit against a second side of the amputee's limb; an articulated hinge having a first member, a middle member, and a second member, the first member comprising a first plate connected to a second plate at an angle to each other, with the second plate hingedly connected to a first end of the middle member and the second member hingedly attached at a second end of the middle member, with the first plate removably connected to the base and the second member of the articulated hinge attached to the second shell; the first plate further having fastening holes configured to align with positioning holes in the base to adjust the relative anterior/posterior position of the first plate and the base prior to connection, the fastening holes and positioning holes configured to accommodate removable fasteners; and the articulated hinge connected at the first member to the base of the first shell and connected at the second member to the second shell forms a space between the first shell and the second shell, the space to be varied to hold, and accommodate size, swelling, and shrinking of the amputee's limb.

These and other objects, features, and advantages of this disclosure will become apparent from the following detailed description of the various aspects of the disclosure taken in conjunction with the accompanying drawings.

### BRIEF DESCRIPTION OF THE DRAWINGS

FIG. 1, is a side view of an embodiment of the post-operative interface socket of the present invention;

FIG. 2 is a top view of an embodiment of the post-operative prosthetic socket with the front and rear shells spread apart;

FIG. 2A is a rear view of an embodiment of the post-operative interface socket showing the structure and connection of the posterior or rear strut among other things;

FIG. 2B is a view of the front of an embodiment of the post-operative interface socket showing among other things the structure of the front connecting strut;

FIG. 2C is a view of the bottom of the post-operative interface socket;

FIG. 3 provides a front left side view of an embodiment of the post-operative interface socket;

FIG. 3A provides a top view of an embodiment of the post-operative interface socket in its fully open or dilated position;

FIG. 3B provides a top view of an embodiment of the post-operative interface socket in its intermediate closed position;

FIG. 3C provides a top view of an embodiment of the post-operative interface socket in its fully constricted position;

FIG. 3D is a view of a loop connecting pad laid out;

FIG. 4 provides a side perspective view of an embodiment of the post-operative interface socket with securing devices attached, including a loop connecting pad;

FIG. 5 is a front left side view of an embodiment of the post-operative interface socket of the present invention with the upper rear shell removed;

FIG. 6 is a rear right-side view of an embodiment of the post-operative interface socket with the upper rear shell removed;

FIG. 6A is a schematic view of the right side of an embodiment of the post-operative interface shell of the present invention which depicts a supra condylar supra patellar (SC SP) connecting trim line;

FIG. 6B is a schematic view of the right side of an embodiment of the post-operative interface shell of the present invention which depicts a supra condylar (SC) connecting trim line;

FIG. 6C is a schematic view of the right side of an embodiment of the post-operative interface shell of the present invention which depicts a patellar tendon bearing (PTB) connecting trim line;

FIG. 7 is a front view perspective view of an embodiment of the outer or tower socket;

FIG. 8 is a side perspective view of an embodiment of the outer or Lower socket of the present invention in a constricted position;

FIG. 8A is the same view as FIG. 8 but with the back shell of the lower socket at a fully open position;

FIG. 9 is a rear perspective view of an embodiment of the outer or Lower socket of the present invention;

FIG. 10 is a slightly raised perspective front left side view of an embodiment of the combined post-operative interface socket and tower or outer socket system as they would appear connected;

FIG. 11 is the same view as FIG. 10 with the addition of a securing patch and an amputee's leg visible;

FIG. 12 is a left side view of an embodiment of the combined post-operative interface socket and lower or outer socket with the upper rear shell removed from the lower rear shell of the post-operative interface socket to thereby allow the knee of the amputee to flex and bend;

FIG. 13 is a side view of an alternative embodiment of the outer or lower socket of the present invention;

FIG. 14 is a is a side view or outer or lower socket depicted in FIG. 13 holding the upper post-operative interface socket that does not have an upper back shell; and;

FIG. 15 is a side view of the lower socket of FIG. 13 holding a post-operative interface socket with an upper back shell.

FIG. 16A a top or plan view of an articulated socket attachment plate system;

FIG. 16B a side view of an articulated socket attachment plate system;

FIG. 17A is a side raised view of the lower or outer socket with the articulated socket attachment installed;

FIG. 17B is a back view of the lower or outer socket with the articulated socket attachment installed; and

FIG. 17C is a side view of the lower socket and its rear shell positioned to accommodate a reduction in size of the upper interface socket; and

FIG. 18 is a side view of a front shell of an embodiment of the invention that has a single socket; and

FIG. 19 is a side view of the rear shell of the variation of the invention that has a single socket; and

FIG. 20 is a side view of the single socket embodiment prosthetic system; and

FIG. 21 is a side view of the single socket prosthetic system showing how the rear shell can pivot forward to accommodate changes in the size of the amputated limb during recovery;

FIG. 22 depicts the single socket prosthetic system with the upper portion of the rear shell removed along a trim line after post-operative recovery to allow the amputee to flex his or her leg;

FIG. 23 depicts the single socket prosthetic system with the upper portion of the rear shell and front shell removed along another trim line after post-operative recovery to allow the amputee to flex his or her leg;

FIG. 24 is a schematic diagram of a variation of a system that uses wedges to position and secure the position of the front shell and rear shell when they are connected by an articulated hinge;

FIG. 25A is a side view of the articulated socket plate system, in accordance with one or more embodiments set forth herein;

FIG. 25B is a second side view of an articulated socket plate system of FIG. 25A with a cap and a wedge, in accordance with one or more embodiments set forth herein;

FIG. 25C is a front perspective view of the articulated socket plate system of FIG. 25B with the cap affixed, in accordance with one or more embodiments set forth herein;

FIG. 25D is a rear perspective view of the articulated socket plate system of FIG. 25B, in accordance with one or more embodiments set forth herein;

FIG. 26A is side view of a plate member of the articulated socket plate system of FIG. 25A, in accordance with one or more embodiments set forth herein;

FIG. 26B is a perspective view of the plate member of FIG. 26A, in accordance with one or more embodiments set forth herein;

FIG. 26C is a top view of the plate member of FIG. 26A, in accordance with one or more embodiments set forth herein;

FIG. 27A is a front perspective view of an upper plate of the articulated socket plate system of FIG. 25A, in accordance with one or more embodiments set forth herein;

FIG. 27B is a side view of the upper plate of FIG. 27A, in accordance with one or more embodiments set forth herein;

FIG. 27C is a bottom perspective view of the upper plate of FIG. 27A, in accordance with one or more embodiments set forth herein;

FIG. 28A is a side view of a middle plate of the articulated socket plate system of FIG. 25A, in accordance with one or more embodiments set forth herein;

FIG. 28B is a perspective view of the middle plate of FIG. 28A, in accordance with one or more embodiments set forth herein;

FIG. 28C is a top view of the middle plate of FIG. 28A, in accordance with one or more embodiments set forth herein;

FIG. 28D is a front view of the middle plate of FIG. 28A, in accordance with one or more embodiments set forth herein;

FIG. 29A is side view of a wedge of the articulated socket plate system of FIG. 25A, in accordance with one or more embodiments set forth herein;

FIG. 29B is a rear perspective view of the wedge of FIG. 29A, in accordance with one or more embodiments set forth herein;

FIG. 29C is a rear view of the wedge of FIG. 29A, in accordance with one or more embodiments set forth herein;

FIG. 29D is a bottom perspective view of the wedge of FIG. 29A, in accordance with one or more embodiments set forth herein;

FIG. 30A is a side view of the cap of FIG. 25A, in accordance with one or more embodiments set forth herein;

FIG. 30B is a rear perspective view of the cap of FIG. 25A, in accordance with one or more embodiments set forth herein;

FIG. 30C is a top rear perspective view of the cap of FIG. 25A, in accordance with one or more embodiments set forth herein;

FIG. 30D is a bottom view of the cap of FIG. 25A, in accordance with one or more embodiments set forth herein;

FIG. 30E is a side cross-sectional view of the cap of FIG. 25A of section E-E of FIG. 30C, in accordance with one or more embodiments set forth herein;

FIG. 31 is a side view of a configuration of the articulated socket plate system of FIG. 25A, in accordance with one or more embodiments set forth herein;

FIG. 32 is a side view of an alternate configuration of the articulated socket plate system of FIG. 25A, in accordance with one or more embodiments set forth herein;

FIG. 33A is a depiction of a side cross sectional side view of a configuration of the articulated socket plate system of FIG. 25A within the single socket embodiment prosthetic system of FIG. 20, in accordance with one or more embodiments set forth herein;

FIG. 33B is a depiction of a side cross sectional side view of a configuration of the articulated socket plate system of FIG. 25A with a spacing wedge and within the single socket embodiment prosthetic system of FIG. 20, in accordance with one or more embodiments set forth herein;

FIG. 33C is a depiction of a side cross sectional side view of a configuration of the articulated socket plate system of FIG. 25A within the single socket embodiment prosthetic system of FIG. 20, in accordance with one or more embodiments set forth herein;

FIG. 33D is a depiction of a side cross sectional side view of a configuration of the articulated socket plate system of FIG. 25A with a positioning wedge and within the single socket embodiment prosthetic system of FIG. 20, in accordance with one or more embodiments set forth herein;

FIG. 33E is a depiction of a side cross sectional side view of a configuration of the articulated socket plate system of FIG. 25A with a positioning wedge and within the single socket embodiment prosthetic system of FIG. 20, in accordance with one or more embodiments set forth herein;

FIG. 33F is a depiction of a side cross sectional side view of a configuration of the articulated socket plate system of FIG. 25A with a positioning wedge and within the single socket embodiment prosthetic system of FIG. 20, in accordance with one or more embodiments set forth herein;

FIG. 34A is a side view of the spacing wedge of FIG. 33B, in accordance with one or more embodiments set forth herein;

FIG. 34B is a top view of the spacing wedge of FIG. 33B, in accordance with one or more embodiments set forth herein;

FIG. 35A is a bottom perspective view of the of the articulated socket plate system of FIG. 25A within a single socket embodiment prosthetic system of FIG. 20, in accordance with one or more embodiments set forth herein;

FIG. 35B is a bottom perspective view of the of the articulated socket plate system of FIG. 25C within a single socket embodiment prosthetic system of FIG. 20, in accordance with one or more embodiments set forth herein;

FIG. 36A is a depiction of a side cross sectional side view of a first sizing configuration of the articulated socket plate system of FIG. 25A with a joint stabilizer cap and within the single socket embodiment prosthetic system of FIG. 20, in accordance with one or more embodiments set forth herein;

FIG. 36B is a depiction of a side cross sectional side view of a second sizing configuration of the articulated socket plate system of FIG. 36A, in accordance with one or more embodiments set forth herein;

FIG. 36C is a depiction of a side cross sectional side view of a third configuration of the articulated socket plate system of FIG. 36A, in accordance with one or more embodiments set forth herein;

FIG. 36D is a depiction of a side cross sectional side view of a fourth configuration of the articulated socket plate system of FIG. 36A, in accordance with one or more embodiments set forth herein;

FIG. 36E is a depiction of a side cross sectional side view of a first configuration of the articulated socket plate system of FIG. 25A with a positioning wedge and within the single socket embodiment prosthetic system of FIG. 20, in accordance with one or more embodiments set forth herein;

FIG. 36F is a depiction of a side cross sectional side view of a second configuration of the articulated socket plate system of FIG. 36E, in accordance with one or more embodiments set forth herein;

FIG. 36G is a depiction of a side cross sectional side view of a third configuration of the articulated socket plate system of FIG. 36E, in accordance with one or more embodiments set forth herein; and

FIG. 36H is a depiction of a side cross sectional side view of a fourth configuration of the articulated socket plate system of FIG. 36E, in accordance with one or more embodiments set forth herein.

### DETAILED DESCRIPTION OF THE INVENTION

The present invention will be discussed hereinafter in detail in terms of various exemplary embodiments according to the present invention with reference to the accompanying drawings. In the following detailed description, numerous specific details are set forth to provide a thorough understanding of the present invention. It will be obvious, however, to those skilled in the art that the present invention may be practiced without these specific details. In other instances, well-known structures are not shown in detail to avoid unnecessary obscuring of the present invention.

Thus, all the implementations described below are exemplary implementations provided to enable persons skilled in the art to make or use the embodiments of the disclosure and are not intended to limit the scope of the disclosure, which is defined by the claims. As used herein, the word “exemplary” or “illustrative” means “serving as an example, instance, or illustration.” Any implementation described herein as “exemplary” or “illustrative” is not necessarily to be construed as preferred or advantageous over other implementations.

The embodiment of the prosthetic system and apparatus of the present invention may be configured for either the right or left leg, depending on which limb has the trans-tibial amputation. Some of the drawings in this application depict the system and apparatus configured for an amputee that has undergone a trans-tibial amputation of the left leg and some of the drawings in this application depict the system and apparatus configured for an amputee that has undergone a trans-tibial amputation of the right leg. However, those skilled in the art will readily understand that the prosthetic system and apparatus of the present invention for the left leg can be easily configured for the right leg and the prosthetic system and apparatus of the present invention for the right leg can be easily configured for the left leg. Furthermore, the version for the left or right legs is essentially mirror images of each other.

Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

I. The Post-Operative Interface Socket of the Perpetual Dimension Prosthetic System

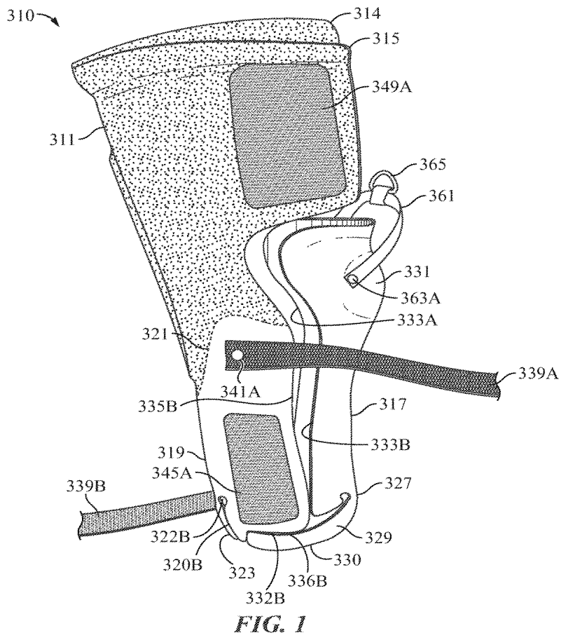

FIG. 1 provides a right side or lateral view of the post-operative interface socket 310 of the present invention. This is the interface socket that an amputees' leg will be inserted into shortly after undergoing a trans-tibial amputation. It includes the upper rear shell 311 or upper anterior shell with left side wing 314 or medial side wing and right side wing 315 or lateral side wing. (References to right or lateral and left or medial herein are from the perspective of the amputee that would be using the prosthetic device, such as the amputee would be facing in the same direction as the front of the prosthetic apparatus faces.

Also, in FIG. 1 tower front shell 317 pr tower anterior shell and lower back shell 319 or lower posterior shell are visible. Upper rear shell 311 detachably connects to lower rear shell 319 along line 321. At its lower end tower back shell 319 connects by posterior or rear flexible strut 323 to base 330 of bottom cup 329. Front shell 317 connects to bottom cup 329 by front strut 327. Dome 331 provides space to accommodate the knee of the amputee.

As appears in FIG. 1 securing strap 339A connects by rivet 341A to the side of lower back shell 319 and a portion of securing strap 339A is visible, it attaches by rivet 341A (not shown in this drawing) to the side of tower rear shell 319. Hook fastener patch 349A is visible on right side wing 315. Hook fastener patch 345A is visible on lower rear shell 319. The full length of the straps is not shown just portions of the straps. In the embodiment of the invention disclosed herein the securing and connecting mechanisms, the straps, patches etc., used are a Velcro®, like fabric hook and Loop connecting system, the hooks being one of the connecting surfaces and the loops on another. Reference will be made to fabric hooks and loop fasteners and will be referred to as the hook and loop fastener or individually as hook fastener or loop fastener. Alternatively, they may be simply referred to as a securing straps or patches. It is noted that any other type of fastener or securing system that can accomplish the same functions can be used.

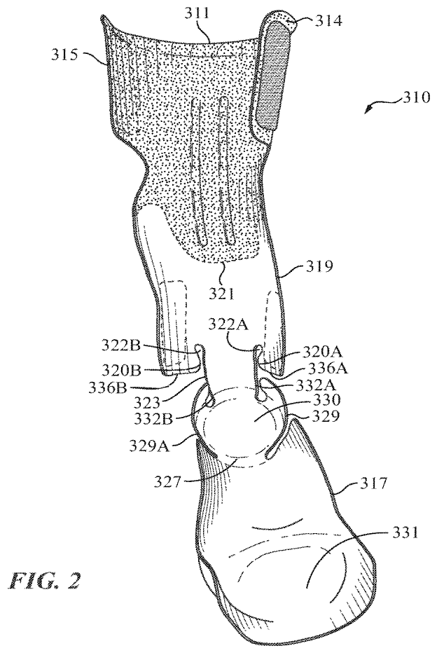

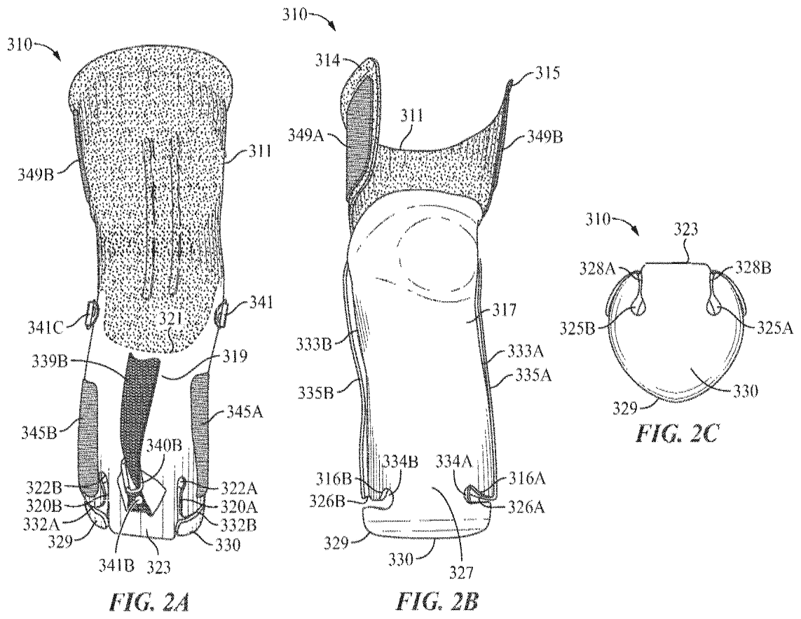

FIG. 2 provides a view of the post-operative interface socket 310 of the present invention with front shell 317 and rear lower shell 319 upper rear shell 311 combination spread out for illustrative purposes. Lower back shell 319 connects to the base 330 of bottom cup 329 by posterior or rear flexible strut 323. Front shell 317 connects to bottom cup 329 by front strut 327, Dome 331 provides a space to accommodate the amputee's knee. FIG. 2 also demonstrates the fact that interface socket 310 is made out of a very flexible but resilient material. In the embodiment depicted polyethylene is the preferred material, but any similar material that has the same qualities can be used.

FIG. 2A is a view of the posterior or rear of post-operative interface socket 310. Upper rear shell 311 detachably connects along line 321 to lower rear shell 319 and back rear flexible strut 323 connects lower rear shell 319 to bottom cup 329. Hook patches 345A and 345B are visible on lower rear shell 319. As noted, above the version of the post-operative interface socket 310 depicted is for the left leg. As noted, posterior or rear flexible strut 323 connects to base 330 of bottom cup 329 at one end and it connects at its opposite end to lower rear shell 319.

For another view we refer to FIG. 2C, a bottom view of post-operative interface socket 310, where open ended slots 328A and 328B eliminate the need for strut 327 to connect to rim 329A of cup 329 and thus can bypass it and attached to bottom 330 of cup 329. By varying the length of slots 328A and 328B the actual connection of strut 323 to base 330 can be made at any selected spot. Open ended slots 328A and 328B end in circular apertures 325A and 325B. The circular apertures 325A and 325B along with slots 328A and 328B add greater flexibility to the system to vary the distance between front shell 317 and lower rear shell 319 which will be discussed below.

Referring back to FIG. 2A rear flexible strut 323 does not connect to the bottom edges 336A and 336B of lower back or rear shell 319 rather as a result of open ended slots 320A and 320B strut 323 attaches further up to lower back shell 319. Here again the length of slots 320A and 320B can be varied to allow the actual point of connect of strut 323 to lower rear shell 319 to be at any desired position on lower back shell 319. Open ended slots 320A and 320B end in circular apertures 322A and 322B. Additionally slots 332A and 332B separate cup 329 from bottom edges 336A and 336B of lower back shell 319. The unique structure described above allows for the movement of lower back shell 319 with respect to front shell 317 to thereby allow of the adjustment of the space between the front shell and the lower back shell and thus accommodate amputated limbs of varying sizes and well as changes in the size or circumference of an amputee's limb that occur overtime during the recovery process as will be discussed further below.

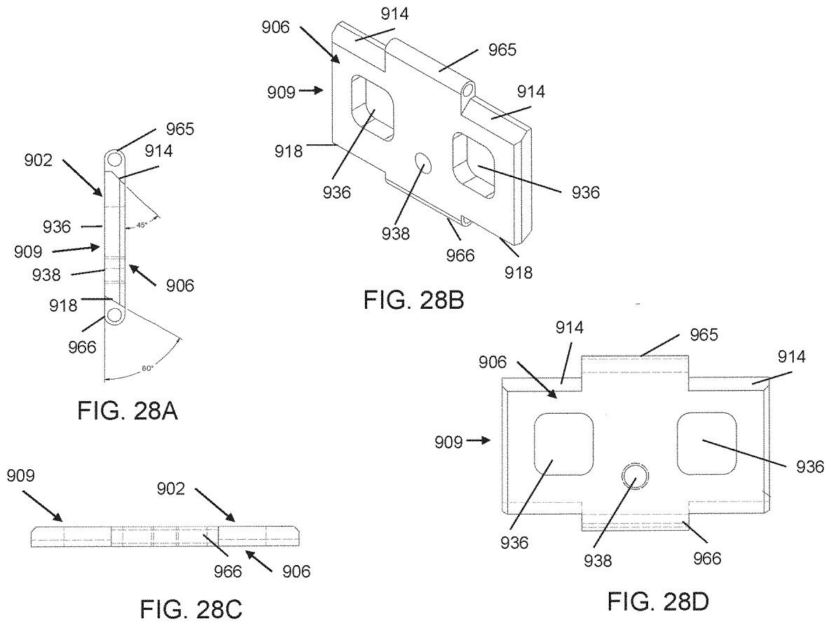

Referring to FIG. 28 an anterior or front view of post-operative interface socket 310. Front shell 317 connects by front connecting strut 327 to cup 329. Front strut 327 connects front shell 317 to rim 329A of bottom cup 329. Strut 327 is narrower than front shell 317, there being open ended slots 326A and 326B on either side of strut 327 that separate and create a space between rim 329A of bottom cup 329 and bottom edges 316A and 316B of front shell 317. Each open ended slot 326A and 326B terminates respectively in circular apertures 334A and 334B. Front shell 317 has side edges 333A and 333B. Also, visible in FIG. 2B are side edges 335A and 335B of lower back shell 319. As will be discussed below given the structure described in this paragraph and depicted in FIG. 2B the side edges 333A and 333B of front shell 317 can be inserted inside of side edges of 335A and 335B of lower back shell 319 to provide an additional means for adjusting the distance between front shell 317 and lower rear shell 319 to vary the space between them and accommodate limbs of different size and also to accommodate changes in the size of the amputated limb held. One of the features of the slot structure and front strut 327 is that it allows for the bending of edges 333A and 333B of front shell 317 to fit inside of edges 335A and 335B to allow for adjusting the space between front shell 317 and lower rear shell 319 by allowing the front shell to slip inside of the lower rear shell.

As noted in a preferred embodiment, post-operative interface socket 310 is made of polyethylene or some similar flexible but durable formable material. Also, in the embodiment depicted, its thickness is approximately 2 to 3 millimeters.

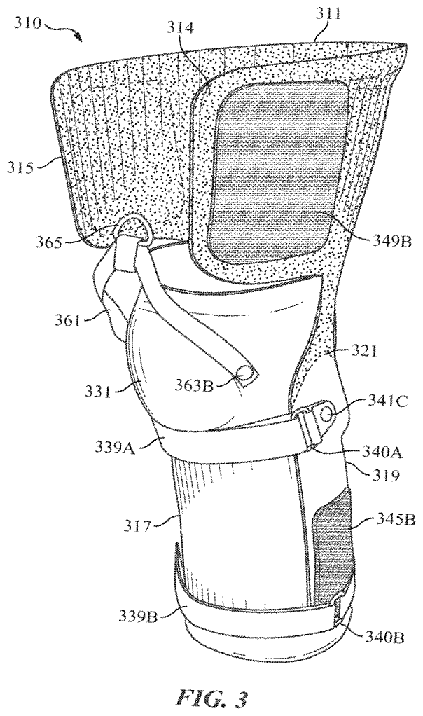

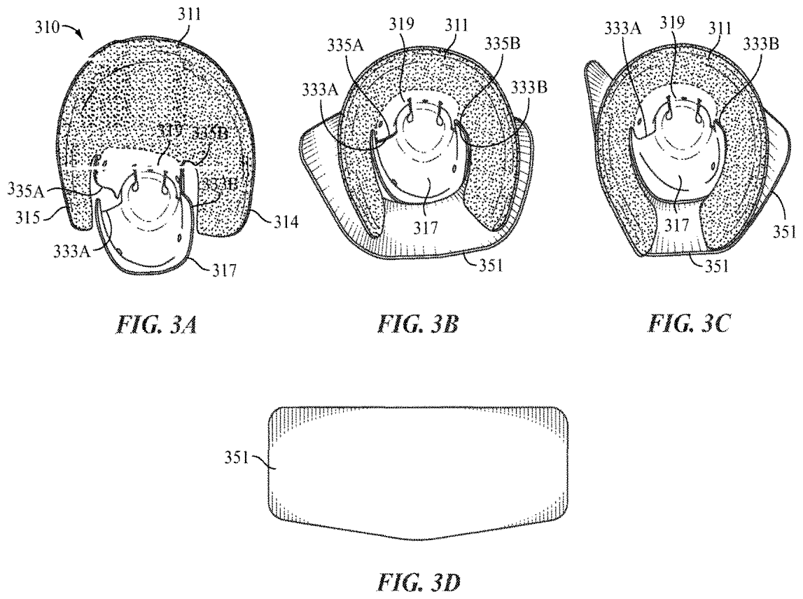

FIG. 3 provides a front left side or front medial side perspective view of post-operative interface socket 310 of the present invention. In this view straps 339A and 339B have been secured around front shell 317. Strap 339A has been looped through metal loop 340A and then secured back on itself with hook and loop connectors it has on opposing surfaces. Loop 340A being secured by rivet and fabric strap 341C. Likewise strap 339B has been secured around front shell 317. Strap 339B has been looped through loop 3408 and then secured back on itself with hook and loop connectors it has on opposing surfaces. As depicted in FIG. 2A strap 339B is connected to the back of lower rear shell 319 by rivet 341B. Referring back to FIG. 3 Strap 361 is secured at one end by rivet 363B and metal and metal loop 365 is at the center of strap 361. Hook securing patch 349B is on the outside of left side wing 314 of upper rear shell 311. Hook securing patch 345B is on lower rear shell 319. It should be noted that strap 339B is covering the slots on lower rear shell 319 and front shell 317 so they are not visible.

FIGS. 3A, 3B, and 3C are top views of the post-operative interface socket 310 that depict the variations of its interior diameter, which can vary to accommodate limbs of varying size and also adjust for swelling or decrease in the size of a limb that normally occurs during the post-operative recovery period.

FIG. 3A provides a top view of post-operative interface socket 310 in the fully open position with the edges 333A and 333B of front shell 317 separated from edges 335A and 335B of lower back shell 319. In FIG. 38, the interior diameter formed by front shell 317 and lower rear shell 319 is decreased and with the edges 333A and 333B of front shell 317 abutting against edges 335A and 335B of front shell 317 abutting against edges 335A and 335B of lower bottom shell 319. In FIG. 3C edges 333A and 333B of front shell 317 have slipped inside of shell 319 further reducing the interior diameter of post-operative interface socket 310. The edges of lower rear shell 319 are not visible because they are covered by the sides of front shell 317. Also, in FIG. 3B loop securing patch 351 has been attached to hook securing patches 349A and 349B (patches 349A and B are visible in FIGS. 1 and 3). Attaching loop securing patch 351 allows one to reduce the curvature of wings 315 and 314 of upper rear shell 311. In FIG. 3C patch 351 has been attached and wings 315 and 314 have been further constricted, FIG. 3D provides a view of fabric loop securing patch laid out.

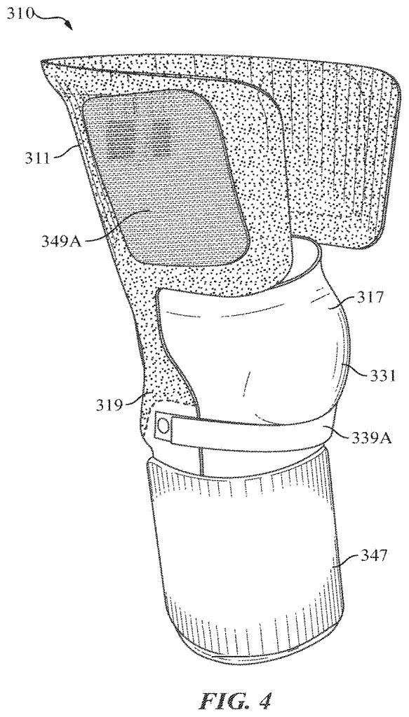

FIG. 4 is a right slightly raised side perspective view of the post-operative interface socket of the present invention with securing devices attached. Strap 339A is secured around front shell 317 and loop securing patch 347 is attached to hook patches 345A and 345B securing it around front shell 317. Hook patches 345A and 345B are visible in FIGS. 1 and 3. Strap 339B is not visible since it is covered by patch 347.

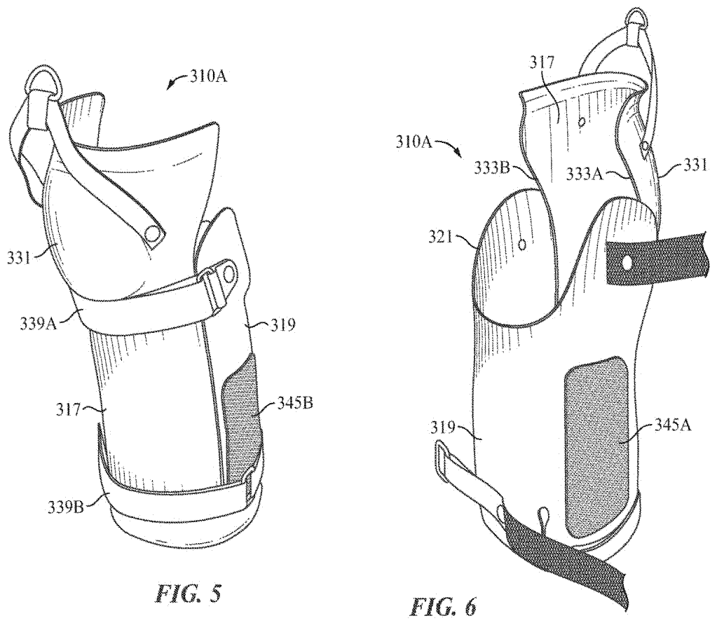

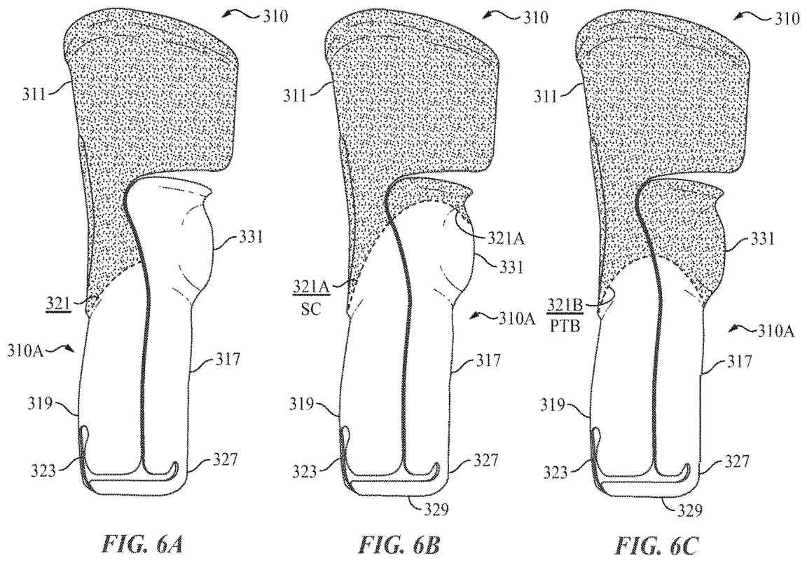

Referring to FIGS. 1, 2 and 2A connecting line 321 along which upper rear shell 311 which is a detachable connection to lower rear shell 319 is in effect a trim line. The type of trim line depicted in FIG. 5 and FIG. 6 after upper rear shell 311 is removed is a supra condylar supra patellar (SCSP) trim line. Note that upon removal of upper rear shell 311 the interface socket is identified by reference number 310A in this specification. However, the trim line can be varied depending on the needs of the particular amputee. FIGS. 6A, 6B, and 6C provide a schematic diagram of some of the various trim lines that can be achieved with the present invention. In FIGS. 6A, 6B and 6C the hatched area in each figure is the portion of post-operative interface shell 310 namely upper rear shell 311 that is removed after the amputee's leg has sufficiently healed such that the leg and specifically the knee no longer needs to be immobilized and it becomes interface socket 310A.

FIG. 6A is an SCSP trim line wherein upper rear shell 311 is removed along SCSP trim line 321. As can be seen only upper rear shell 311 is removed. FIG. 6B depicts a supra condylar (SC) trim line 321A wherein not only is rear shell 311 detached along line 321A but trim line 321A continues along the top of front shell which is also removed. FIG. 6C shows a patellar tendon bearing (PTB) trim line 321B where upper rear shell 311 is detached from lower rear shell 319 along line 321B, and trim line 321B continues along front shell 317 to thereby remove a significant portion of front shell at and below where the knee of the amputee would be when the amputee is wearing the post-operative interface socket 310A.

As demonstrated in FIGS. 3A, 3B, and 3C above by use of straps 339A and 339B the interior diameter of post-operative interface socket 310 can be varied as depicted in FIGS. 3A, 38, and 3C. By placing security hook fastener patch 347 around front shell 317 connected to patches 345A and 345B, this provides an additional means for securing a desired or required inner space in post-operative interface socket 310 and to adjust it to the varying size of the post-operative trans-tibial amputated limb.

The ability to vary the interior circumference of post-operative interface socket 310 and likewise 310A, after removal of upper shell 311, provides a number of advantages. First, it reduces the number of sizes of post-operative interface sockets 310 that need to be maintained in stock to accommodate amputee limbs of varying size. The flexibility in sizing described above increases the dimensional range of the anterior-posterior (AP) range and medial-lateral (ML) and circumferences from 4″ to 6″ depending on the size of the patient. Additionally, the circumference of the portion of amputated limb will vary significantly from the time of the operation amputating the lower limb to the time the amputee is able to walk normally. Right after the operation, the limb will typically swell due to the trauma of the surgery. While recovering from the operation and healing, the amputee's limb will shrink in diameter as part of the healing process as well as the relative inactivity imposed on it. This is in part due to the need to keep the knee immobilized during the initial recovery phase. Post-operative interface socket 310 can be easily adjusted by straps 339A and 339B and patch 347.

Once the initial healing process is complete and the patient can start flexing their knee, there is no need to switch to another interface socket. As depicted in FIG. 5, upper back or rear shell 311 is detached from lower back shell 319 along line 321. This then allows the amputee to continue to use post-operative interface socket 310A and start to flex his or her knee. Upper back shell 311 when attached to lower back shell 319 is designed to immobilize the limb and prevent flexing of the knee to aid in the initial heating process. However, once the healing process has progressed sufficiently the amputee needs to start flexing the knee to begin the process of starting to walk and move normally. FIG. 6 provides a rear view of post-operative interface socket 310A with upper rear shell removed so the amputee can flex their knee and bend their leg.

II. The Outer or Lower Socket of the Perpetual Dimension Prosthetic System

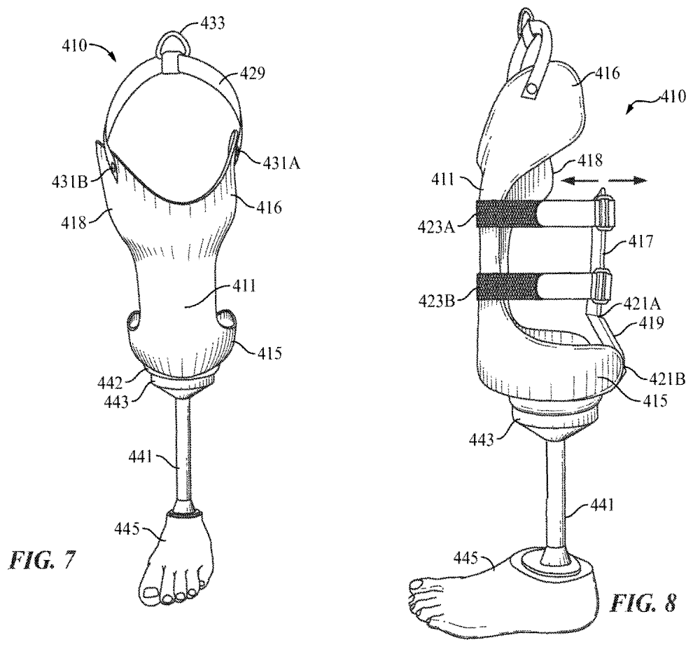

Outer or lower socket 410 is depicted in FIGS. 7, 8 and 9 in a front view, side view and rear view respectively. In a preferred embodiment polypropylene is the preferred material for outer socket 410. It provides a flexible, sturdy, resilient, formable, and lightweight material. In the embodiment shown the thickness of the material is about 4 to 5 millimeters.

The parts visible in FIG. 7 include front shell 411 or anterior shell, which attaches to base or bottom cup 415. Front shell 411 has a left wing 416 or medial wing and a right wing 418 or lateral wing. (As noted previously references to right and left herein are from the perspective of the amputee that would be using the prosthetic device, such as the direction the front of the object described faces). Strap 429 connects to wing 418 by rivet 431B and to wing 416 by rivet 431A. Retention ring 433 is attached to the center of strap 429. Attached to the bottom receiving plate 442 which forms a reinforced base of bottom cup 415 is hardware connecting device 443, shaft 441, and artificial foot 445. In a standard fashion, bolts (not shown) would pass up through hard connecting device 443, through receiving plate 442 and connect to a retention bracket on the inside of bottom of outer socket 410. Shaft 441 securely attaches in a standard fashion to connecting hardware device 443. The connection created is strong enough to bear the weight and stress and strain of an adult waking the prosthetic system. Artificial foot 445 completes the system.

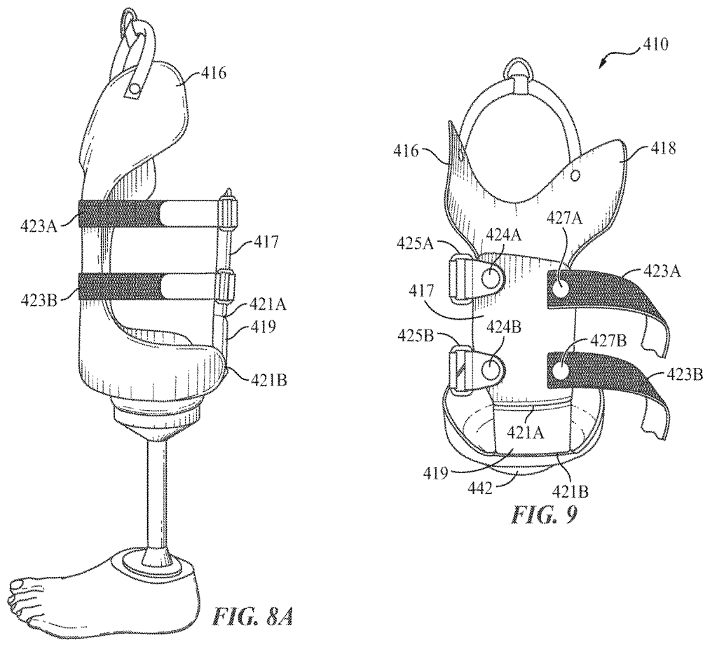

FIG. 8 provides a side view of lower or outer socket 410. In this side view additional parts of outer or lower shell are visible, namely back shell 417 or posterior shell and articulated strut 419 that connects to back shell 417 at flex line 421A and to base cup 415 at flex line 421B. Securing straps 423A and 423B connect around front shell 411 and hold back shell 417 in position.

FIG. 9 is a rear view or posterior view of outer socket 410 without items 443, 441 and 445 attached. Additional aspects of lower socket 410 visible are flex lines 421A and 421B that allow articulate strut 419 to move back and forth. Rivet 427A holds strap 423A to back shell 417 and rivet 427B holds strap 423B to back shell 417. Rivet 424A holds metal loop and retention strap 425A and rivet 424B holds metal loop and retention strap 425B to back shell 417.

Flex lines 421A and 421B in the embodiment depicted in FIG. 9 are created by compressing the material which lower socket 410 is made of, in the embodiment depicted polypropylene. Flex lines 421A and 421B by movement of articulated strut 419 allow back shell 417 to be moved between the positions depicted in FIG. 8 and FIG. 8A and thus allow outer socket to adjust to the varying circumferential sizes the post-operative interface socket 310 will varying given the different in limb sizes of amputees and the variation of a particular amputee's limb that occurs during the heating and recovery process.

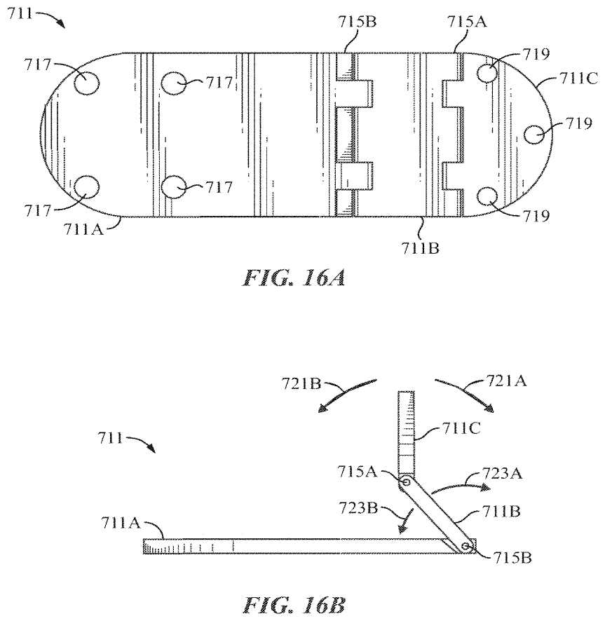

FIG. 16A depicts another variation of the articulated and movable strut (419 depicted in FIGS. 8, 8A and 9). In the variation depicted in FIG. 16A is a separate double hinged socket attachment plate 711. Hinged socket attachment 711 is made up of a lower plate 711A, a middle plate 711B, and an upper plate 711C. Lower plate 711A connects to middle plate 711B by hinge 715B. Middle plate 711B connects to upper plate 711C by hinge 715A. Referring now to FIG. 16B a side view of articulated socket attachment plate 711. As depicted in FIG. 16B, both hinges 715A and 715B are fully articulated allowing the plates to be freely moved with respect to each other. As depicted plate 711C can be pivoted on hinge 715A with respect to plate 711B in the direction of arrows 721A and 721B. Likewise, plate 711B can be pivoted on hinge 715B with respect to its orientation to plate 711A in the direction of arrows 723A and 723B. Plate 711 can be fabricated from any number of rigid but durable materials including aluminum, polypropylene, polyethylene, stainless steel, etc. Screw holes 717 on plate 711A and screw holes 719 on plate 711C provide means for attaching articulated socket attachment 711 to the base cup and the back shell of the outer or lower socket, as will be described below.

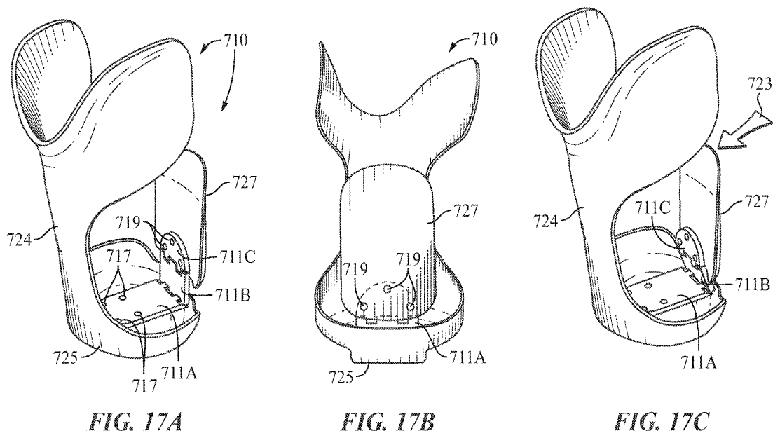

Referring now to FIG. 17A a side raised view of the lower or outer socket 710 with articulated socket plate system 711 attached to the lower or outer socket. As depicted, lower socket attachment plate 711A is attached to the top inside of base cup 725 of the lower socket. Plate 711A is attached in the standard fashion with screws inserted through screw holes 717. Plate 711C is attached to back shell 727, as depicted, in the standard fashion by inserting screws through screw holes 719. In this variation of the lower socket back shell is separate from the rest of the lower socket and only connected by the articulated socket attachment plate 711.

FIG. 17B is a rear view or posterior view of tower socket 710 with articulated plate system 711 attached. As can be seen, back shell 727 is not directly attached to lower cup 725. Rather, articulated plate system 711B connects lower cup 725 to back shell 727. This is to allow for movement of back shell 727 to vary the size of space lower socket 710 provides. It thus serves the same function as articulated strut 417. FIGS. 8 and 8A show how articulated strut 417 allows for the varying of the distance between back shell 417 and the front of lower socket 410. FIG. 17C depicts how rear shell 727 can be moved forward by adjusting plates 711C and 711B with respect to 711A. This thus allows lower socket 710 to accommodate the varying size of the upper interface socket 310 as the trans-tibial amputee proceeds through the recovery process.

III. The Over all Combined Post-Operative Lower Socket Perpetual Dimension Prosthetic System

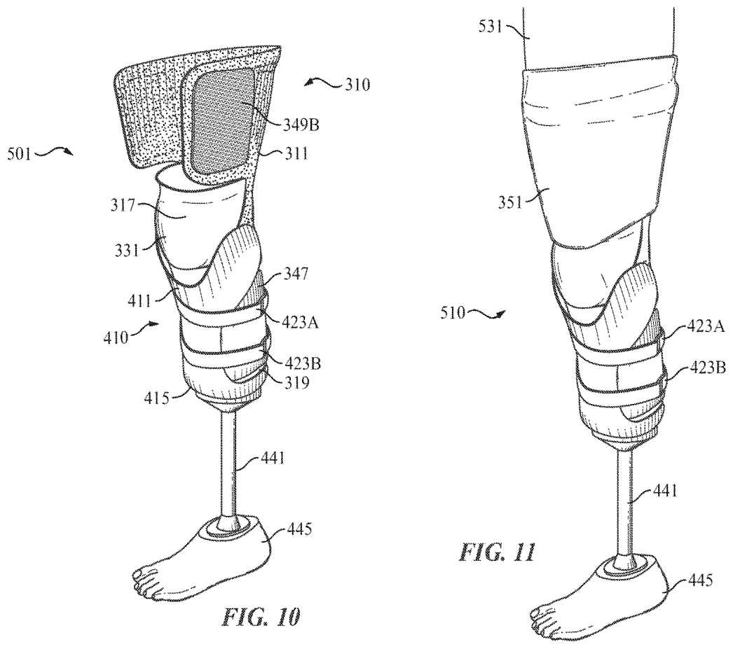

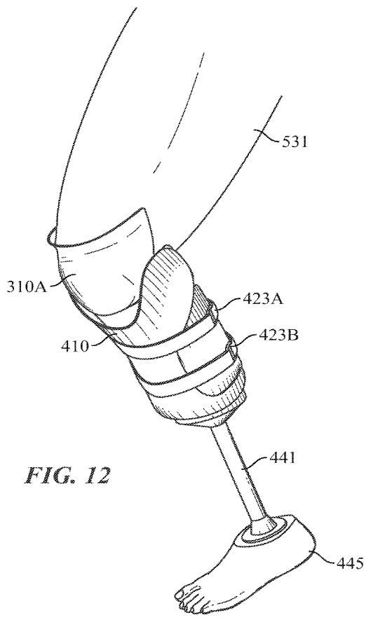

FIG. 10 provides a front left side or anterior lateral side prospective view of the parts of the prosthetic system 501 of the present invention namely post-operative interface socket 310 as it is used with outer lower socket 410. Post-operative interface socket 310 is positioned inside of outer socket 410 with shaft 441 and artificial foot 445 attached in a configuration ready for a trans-tibial amputee to use. FIG. 11 provides a front left side or anterior lateral side perspective view of the prosthetic system 501 of the present invention as it might appear on an amputee's leg 531. The limb 531 of an amputee can be seen and loop patch 351 has been attached to hook pads 349A and 349B. As depicted in FIG. 11 upper rear shell 311 immobilizes the limb 531 and prevents it from flexing at the knee. However, as pictured in FIG. 12, a rear left side view of the entire prosthetic system 501, upper rear shell 311 has been removed to allow limb 531 to flex at the knee as depicted therein.

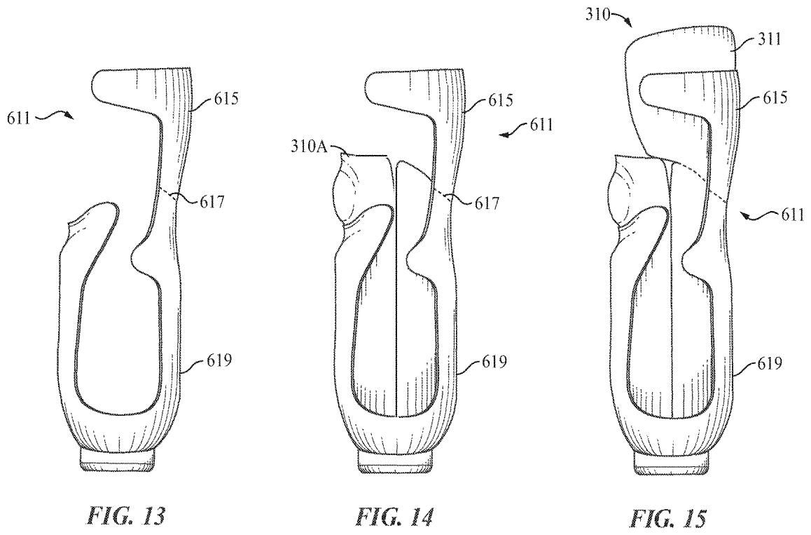

FIG. 13 provides a side view of an alternative version of the tower socket 611. In this variation of the invention lower socket 611 has a detachable upper back shell 615 detachable along line 617. Detachable upper back shell 615 has the same purpose as the detachable upper back shell 311 of the upper or interface socket 310, FIG. 1. Referring back to FIG. 13 upper back shell 615 of lower socket 611 is also designed to immobilize the trans tribal leg amputee's and prevent flexing of the knee during the initial heating process after the amputation. As noted previously the knee of the amputee needs for a period of time after the amputation to recover from the trauma of the operation.

FIG. 14 provides a side view of lower shell 611 of lower socket 611 holding post-operative interface socket 310A. In this variation depicted upper back shell 615 of lower socket 611 takes the place of upper rear shell 311 of interface socket 310. Once the amputee has sufficiently recovered upper shell 615 is removed along line 617. Once this upper shell 615 is removed the amputee then can flex his or her knee as depicted in FIG. 12.

FIG. 15 provides another variation of the invention where upper or post-operative interface socket 310 with upper back shell 311 is used with lower socket 611. In this variation both upper rear or back shell 311 of interface socket 310 and upper shell 615 of lower socket 611 help immobilize the knee of the amputee during the initial healing process. Thus, once the healing process is complete both shell 311 and 615 can be removed to allow the amputee to flex his or her knee.

IV. An Alternative Single Socket Post-Operative Perpetual Dimension Prosthetic System

FIG. 16A depicts an articulated and movable strut 711. In the strut 711 depicted in FIG. 16A is a separate double hinged socket attachment plate. The strut 711 includes the hinged socket attachment having a lower plate 711A, a middle plate 711B, and an upper plate 711C. Lower plate 711A connects to middle plate 711B by hinge 715B. Middle plate 711B connects to upper plate 711C by hinge 715A. Referring now to FIG. 16B a side view of the strut 711 is depicted. Both hinges 715A and 715B are fully articulated allowing the plates (e.g., lower plate 711A, middle plate 711B, and upper plate 711C) to be freely moved with respect to each other. As depicted plate 711C can be pivoted on hinge 715A with respect to plate 711B in the direction of arrows 721A and 721B. Likewise, plate 711B can be pivoted on hinge 715B with respect to its orientation to plate 711A in the direction of arrows 723A and 723B. Plate 711 can be fabricated from any number of rigid but durable materials including, for example, aluminum, polypropylene, polyethylene, carbon fiber and epoxy resin, stainless steel, or similar materials, or combinations of such materials. Screw holes 717 on plate 711A and screw holes 719 on plate 711C provide means for attaching articulated strut 711 to the base cup and the back shell of the outer or lower socket, as will be described below. While four screw holes 717 are depicted, there may be embodiments with less than four screw holes 717 or embodiments with more than four screw holes 717. Also, while screw holes are depicted as a means for attaching strut 711, there may be embodiments where other fastening means such as, for example, rivets, adhesives, or epoxies may be used.

Referring now to FIG. 17A a side raised view of the lower or outer socket 710 with articulated socket plate system 711 attached to the lower or outer socket. As depicted, lower socket attachment plate 711A is attached to the top inside of base cup 725 of the lower socket. Plate 711A is attached in the standard fashion with screws inserted through screw holes 717. Plate 711C is attached to back shell 727, as depicted, in the standard fashion by inserting screws through screw holes 719. In this variation of the lower socket back shell is separate from the rest of the lower socket and only connected by the articulated socket attachment plate 711.

FIG. 17B is a rear view of tower socket 710 with articulated plate system 711 attached. As can be seen, back shell 727 is not directly attached to lower cup 725. Rather, articulated plate system 711B connects lower cup 725 to back shell 727. This is to allow for movement of back shell 727 to vary the size of space lower socket 710 provides. It thus serves the same function as articulated strut 417. FIGS. 8 and 8A show how articulated strut 417 allows for the varying of the distance between back shell 417 and the front of lower socket 410. FIG. 17C depicts how rear shell 727 can be moved forward by adjusting plates 711C and 711B with respect to 711A. This thus allows lower socket 710 to accommodate the varying size of the upper interface socket 310 as the trans-tibial amputee proceeds through the recovery process.

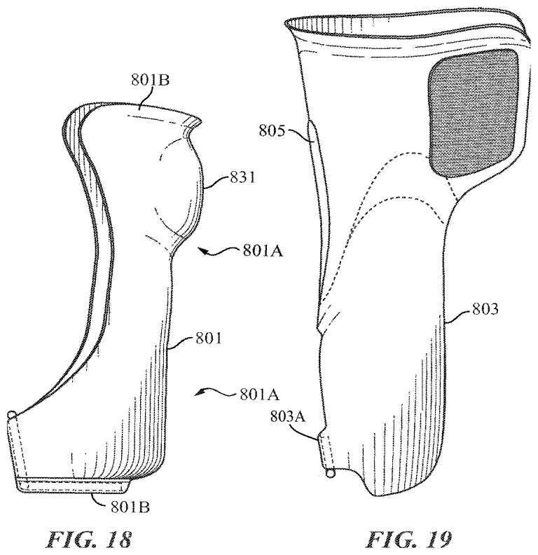

In another embodiment of the invention the prosthetic system consists of a single socket. Thus, the single socket includes configurations (e.g., sizes and dimensions) that provide for post-operation use and for post-recovery use as a prosthetic device. In this embodiment it has a front shell 801 or anterior shell as depicted in FIG. 18. Front shell 801 has an extended surface 801A that is shaped to fit against the front of a trans-tibial amputee's leg or limb. Extended surface 801A of the front shell terminates in a base 801B that is configured (e.g., shaped and dimensioned) to be below the bottom of the amputee's limb. Dome 831 provides room to accommodate the amputee's knee. As discussed below, front shell 801 is configured (e.g., sized and dimensioned) to be the main weight bearing part of the single socket system and is formed of a rigid load bearing material. In the preferred embodiment polypropylene is used to make front shell 801. Polypropylene is easy to work with and form into the desired shape and is strong enough to bear the full Load of an amputee's body when walking or running. Alternate embodiments may use materials of similar rigidity, such as, for example, aluminum, polyethylene, carbon fiber and epoxy resin, stainless steel, or combinations thereof.

FIG. 18 provides a right side view of the socket of the present invention. This is the interface socket that an amputees' leg will be inserted into shortly after undergoing a trans-tibial amputation. FIG. 19 is a side view of an embodiment of a rear shell 803 or posterior shell of the single socket prosthesis of the present invention. As can be seen rear shell 803 has an extended surface configured to fit against the rear of the leg of an amputee. Rear shell 803 is made of a pliable and formable material. In the preferred embodiment it is made of polyethylene, but alternate embodiments may use similar materials, including polypropylene. It includes an upper rear shell 805 with left side wing and right side wing. (References to right and left herein are from the perspective of the amputee that would be using the prosthetic device, such as the amputee would be facing in the same direction as the front of the prosthetic apparatus faces.

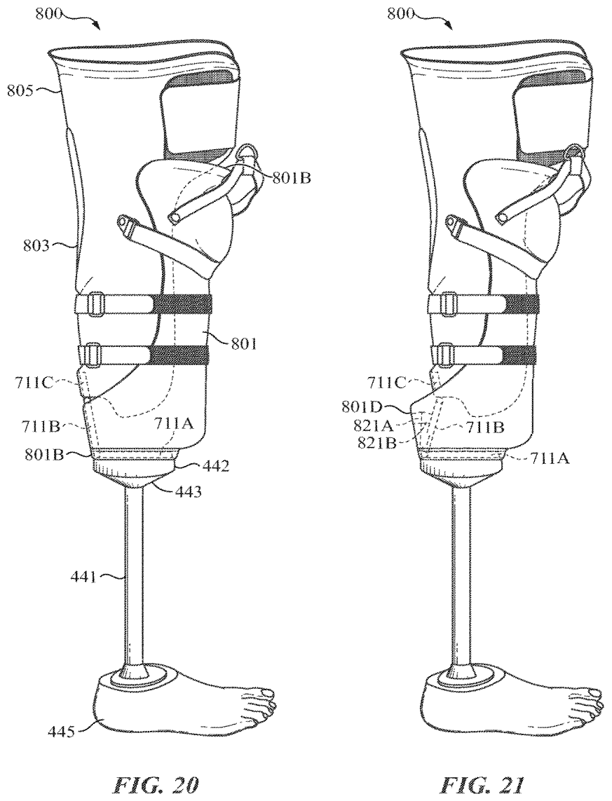

The front shell and rear shell are attached by articulated hinge 711 FIGS. 16A and 16B. Referring now to FIG. 20 which provides a side view of the single socket embodiment 800 of the invention fully assembled. It includes front shell 801 connected to the rear shell 803 by hinge 711 shown in outline since it is inside the shells. Plate 711A attaches to a top inside surface of base 801B and plate 711C attaches to a bottom edge of rear shell 803. Center plate 711B of articulated hinge 711 only connects at pivot points with plates 711A and 711C and thus can move when plates 711A or 711C move.

An artificial foot 445 is connected by a shaft 441 to hardware 443 and a reinforced bottom connection plate 442 of front shell 801. Reinforced connection plate 442 is a reinforced portion of front shell 801 configured (sized and dimensioned) and formed of a material for carrying the load put on it by the amputee when walking or standing.

FIG. 21 shows how the space between the front shell 801 and the rear shell 803 can be varied by articulated hinge 711. As noted above since plate 711B of hinge 711 is only hingedly connected to plates 711A and 711C it can move to allow shell move with respect to shell 801 and change the space between the two shells. As noted previously after an amputation of a limb, the remaining portion of the amputated limb varies in size over the course of the amputee's recovery from the surgery. Initially it will swell as a result of the trauma of the surgery. Subsequently it will also shrink as the patient recovers. It will shrink as a result of the need to minimize use of the limb during recovery. In Particular with a post-operative transitional amputation this includes immobilizing the knee of the amputee until the surgical wound created by the surgery has fully healed.

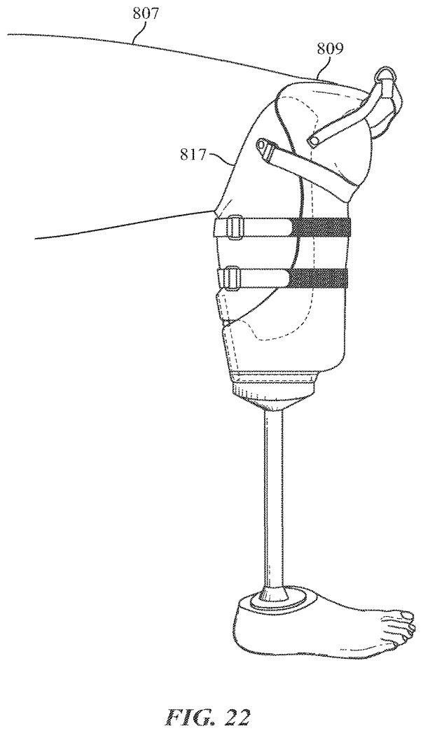

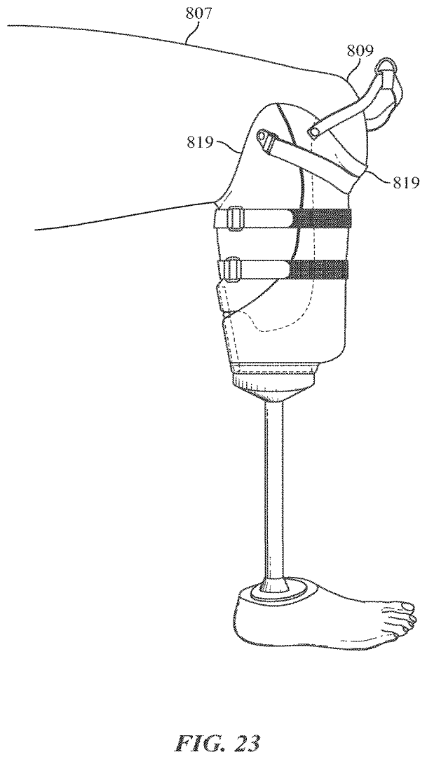

Referring to FIGS. 22 and 23 once the amputee has recovered from the operation and can begin exercising his or her leg the upper portion 805 of the rear shell 803 can be removed to allow the amputee to flex his or her leg 807 and bend the knee and begin therapy to walk in a normal fashion. Additionally, parts of upper portions 801C of the front shell can also be removed. As previously discussed above with respect to the two socket version of the invention the shells can be cut along different trim lines three of which are: 1) Supra Condylar Supra Patellar (SCSP) trim line, 2) super condylar (SC) trim line and 3) Patellar tendon bearing (PTB) trim line (FIGS. 6A, 6B and 6C). Referring back to FIG. 22 it provides an example of a SCSP trim line cut 817 of the single socket system. Likewise, FIG. 23 is an example of PTB trim line cut 819. Naturally, the trim line cut can be varied significantly to achieve optimal movement and support which can include a super condylar (SC) as depicted in FIG. 6B. Thus, any number of variations of cuts can be made depending on the needs of the particular amputee. As depicted in FIGS. 22 and 23 once the trim line cut is made it allows the amputee to move his limb 807 and flex his or her knee 809.

V. An Apparatus for Dimensional Changes of the Socket

In another aspect of the invention, it includes an apparatus for positioning and fixing the dimensional changes of the space between the front and back shells of the sockets that are connected by an articulated hinge. Referring to FIG. 21 as depicted therein two spacing and positioning wedges 821A and 821B are positioned between center plate 711 of articulated hinged 711 and the back rim 801D of base 801B of front shell 801.

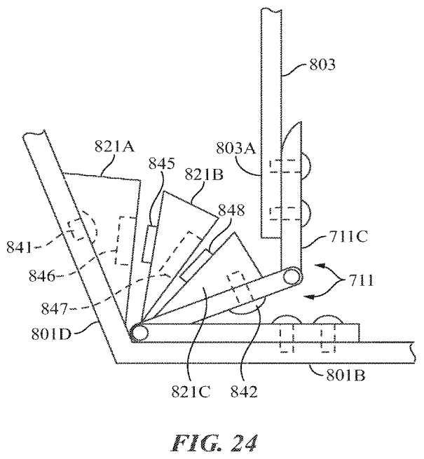

By varying the number of wedges from none to 3, the position of center plate 711B can be fixed. FIG. 24 provides a schematic of the wedge positioning system of the present invention. Articulated hinge 711 connects by plate 711C to a lower portion 803A of rear shell 803. Plate 711A of hinge 711 connects to the inside of bottom base 801B of the front shell Rear rim 801. Spacing wedges 821A, 821B, and 821C are depicted in a semi-exploded view are positioned between rear rim 801D of base 801B of front shell 801 and center hinge plate 711B. In the embodiment depicted in FIG. 24, wedge 821A connects to rear rim 801D by retaining screw 841. Wedge 821C connects to center hinge plate 711B by retaining screw 843. Wedge 821B has knob 845 which fits into recess 846. Wedge 821C has knob 847 which fits into recess 848 of wedge. Thus, when wedges 821A, 821B, and 821C are inserted and connected together they are held in place.

VI. An Alternative Apparatus for Dimensional Changes of the Single Socket Post-Operative Perpetual Dimension Prosthetic System

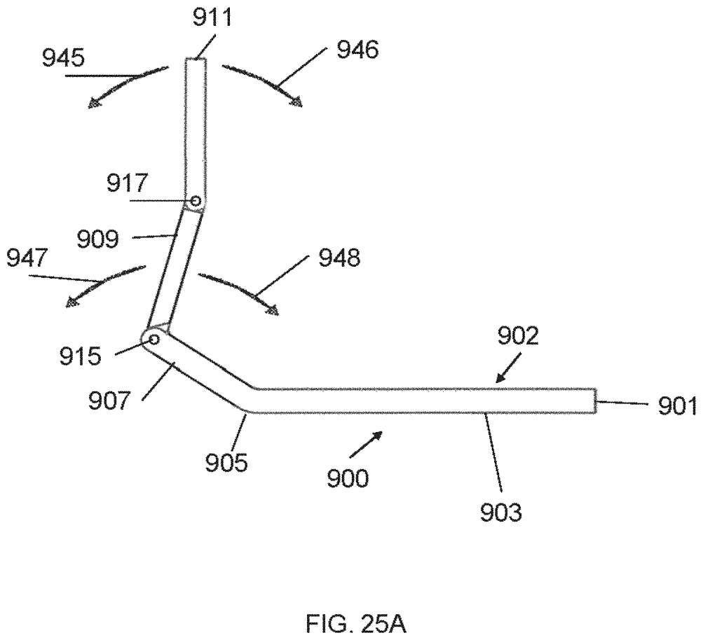

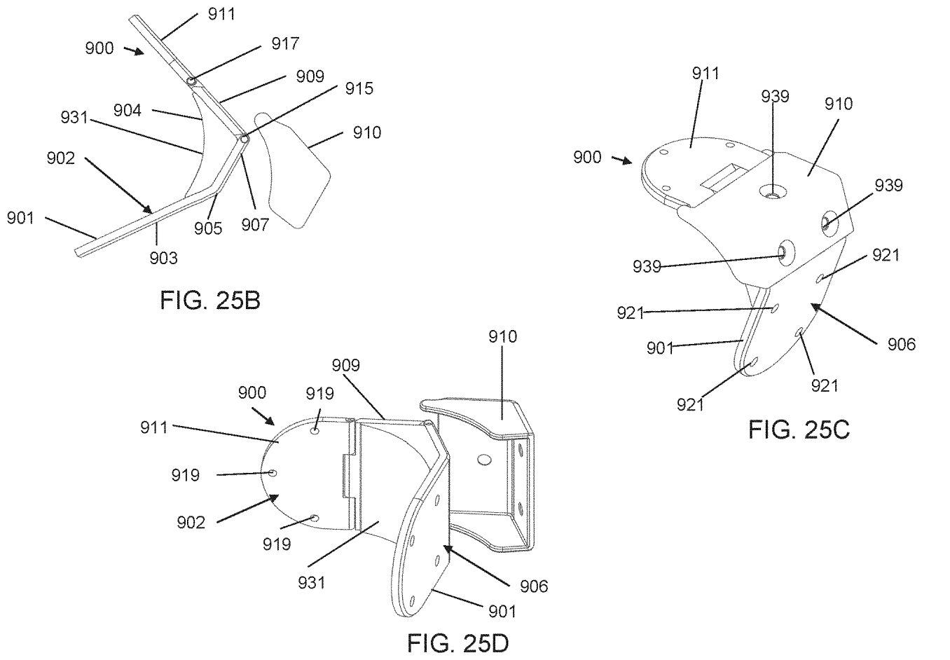

FIG. 25A depicts another variation of the articulated and movable socket attachment (e.g., 419 depicted in FIGS. 8-9 and 711 as depicted in FIGS. 16A, 16B, 20, 21, and 24). An articulated double hinged socket attachment 900 (also referred to as an articulated hinge) is shown in an example of a first configuration. Socket attachment 900 is made up of a plate member 901, a middle plate 909 (also referred to as a center plate or middle member), and an upper plate 911 (also referred to as a second member). Member 901 has a first plate section 903 and a second plate section 907 connected to each other at a connection 905, and at an angle relative to each other. Member 901 connects to middle plate 909 by a first hinge 915, such that member 901 and middle plate 909 may rotate about first hinge 915 relative to each other. First hinge 915 may be at a first end of second plate 907 opposite a second end at connection 905. First hinge 915 is connected to middle plate 909 at a first end and opposite a second hinge 917 at a second end of middle plate 909. Middle plate 909 connects to upper plate 911 by second hinge 917, such that upper plate 911 and middle plate 909 may rotate about second hinge 917 relative to each other.

Both hinges 915 and 917 are fully articulated allowing the plates to be freely moved with respect to each other. As depicted upper plate 911 may be pivoted on hinge 917 with respect to middle plate 909 in the direction of arrows 945 or 946. Likewise, middle plate 909 may be pivoted on hinge 915 with respect to its orientation to member 901 in the direction of arrows 947 or 948. Socket attachment 900 may be fabricated from any number of rigid but durable materials including aluminum, polypropylene, polyethylene, stainless steel, and such similar materials.

With reference to FIGS. 25B-25D, the socket attachment 900 is depicted with a positioning wedge 931 in contact with member 901 and middle plate 909. Also shown is a joint stabilizer cap 910, depicted in contact with member 901 and middle plate 909 on the side opposite wedge 931. When a desired sizing and positioning of socket attachment 900 is found wedge 931 and/or cap 910 may be affixed to socket attachment 900 to hold member 901 and middle plate 909 fixed, relative to each other. Upper plate 911 may still be movable about hinge 917 relative to middle plate 909. Socket attachment 900 has a limb facing surface 902 and an opposite outer surface 906 facing away from the limb. Positioning wedge 931 is positioned on the limb facing side of socket attachment 900.

Referring to FIGS. 18-21 and 25A-D, outer surface 906 of upper plate 911 may be removeably connected to a lower portion of rear shell 803, and surface 906 of middle plate 909 and member 901 may be connected to back rim 801D of base 801B of front shell 801 (similar to how articulated hinge 711 is connected to back rim 801D of base 801B of front shell 801). The position of middle plate 909 relative to member 901 may be fixed using positioning wedge 931 once the desired position is found. Unlike the wedges in FIG. 24, wedge 931 in FIGS. 25A-25D may be limb facing rather than being positioned on the outer surface (e.g., surface 906) and shell facing or base facing.

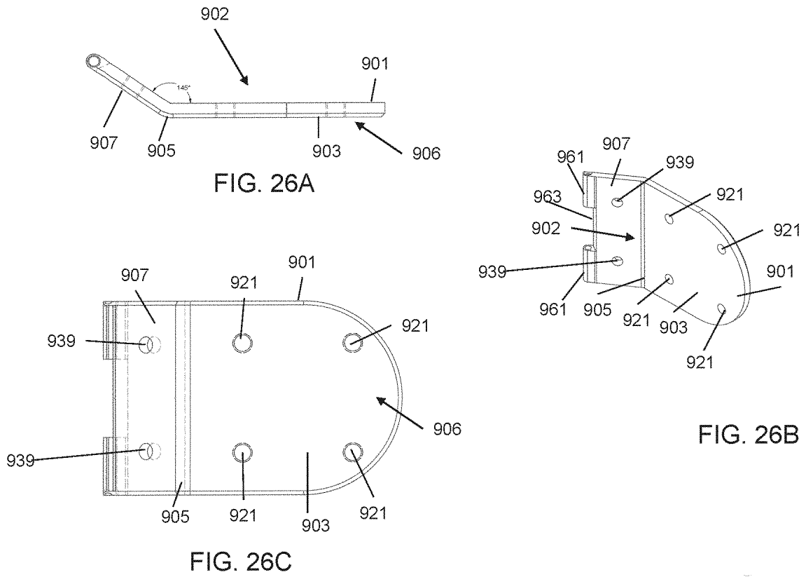

With reference to FIGS. 26A-26C, various view of member 901 are depicted. Member 901 has first plate section 903 and second plate section 907 connected to each other at a connection 905, and at an angle relative to each other. The angle between first plate section 903 and second plate section 907 is shown at approximately 145° as measured on the limb facing surface 902. However, there may be other embodiments with the angle being from approximately 90° to less than 180°. Hinge segments 961 of hinge 915 are depicted at the end of plate 907 opposite connection 905. The hinged segments 961 may be on opposite sides of plate 907 and may provide a space for inserting a hinge segment from the middle plate (e.g., middle plate 909). There may be a sloped extension 963 between the hinge segments 961 to inhibit rotation of middle plate 909 beyond a certain angle. Fastener holes 921 are depicted through plate 901 and fastener holes 939 are depicted through plate 907.

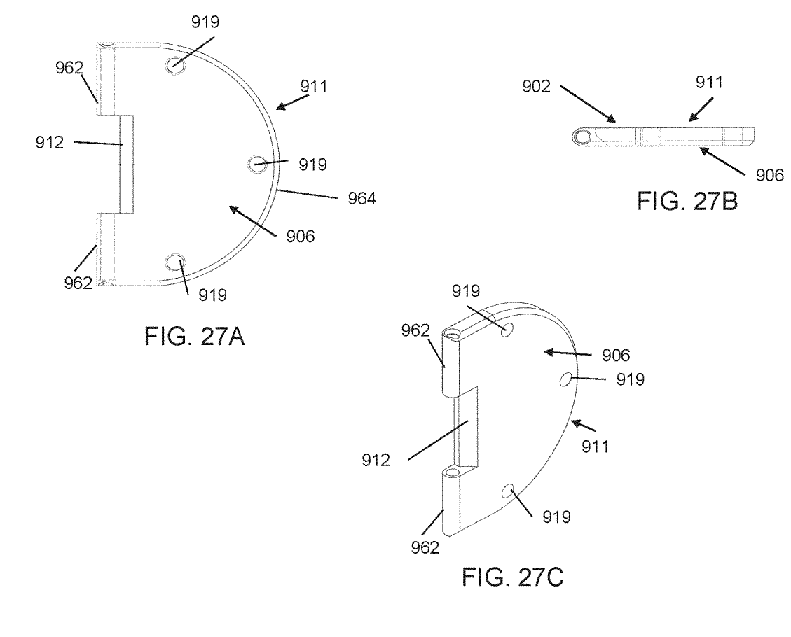

With reference to FIGS. 27A-27C, various view of upper plate 911 are depicted. Upper plate 911 is depicted as being substantially semi-circular in shape, however upper plate may be any shape configured (e.g., shaped and dimensioned) for connection to a middle plate at one end and a rear shell 803. Hinge segments 962 of hinge 917 are depicted at one end of plate 911 opposite a free end 964. The hinged segments 962 may be on opposite sides of plate 911 and may provide a space for inserting a hinge segment from the middle plate (e.g., middle plate 909). There may be a sloped or angled extension 912 between the hinge segments 962 to inhibit rotation of upper plate 911 beyond a certain angle in relation to middle plate 909. Fastener holes 919 extend through plate 911 between the limb facing side 902 and the second side 906.

With reference to FIGS. 28A-28D, various view of middle plate 909 are depicted. The middle plate 909 is a substantially rectangular shape having a hinge connector 966 at a bottom end and configured (e.g., shaped and dimensioned) to form first hinge 915 when connected to hinge segments 961 of second plate 907. Middle plate 909 has a hinge connector 965 at a top end and configured (e.g., shaped and dimensioned) to form second hinge 917 when connected hinge segments 962 of upper plate 911. Angled sections 914 extend along the top from either side of hinge segment 961, sloping from the limb facing side 902 towards a second side 906 facing away from the limb. Angled sections 918 extend along the bottom from either side of hinge segment 966, sloping from limb facing side 902 towards second side 906 facing away from the limb. Angled sections 914 may, for example, inhibit rotation of upper plate 911 beyond a certain angle in relation to middle plate 909. Angled sections 918 may, for example, inhibit rotation of middle plate 909 beyond a certain angle in relation to second plate 907.

Middle plate 909 is depicted as being rectangular, however middle plate 909 may be other shapes in other embodiments.

Middle plate 909 may further have holes 936 extending through middle plate 909 between limb facing side 902 and second side 906. In FIGS. 28B and 28D, holes 936 are depicted as square however, in other embodiments, the holes may be other shapes. While two holes 936 are depicted, there may be more or less than two holes in other embodiments. A screw hole 928 may extend through middle plate 909 and is configured (e.g., shaped and dimensioned to accommodate a fastener. In certain embodiments, screw hole 928 may be threaded to accommodate a screw or bolt.

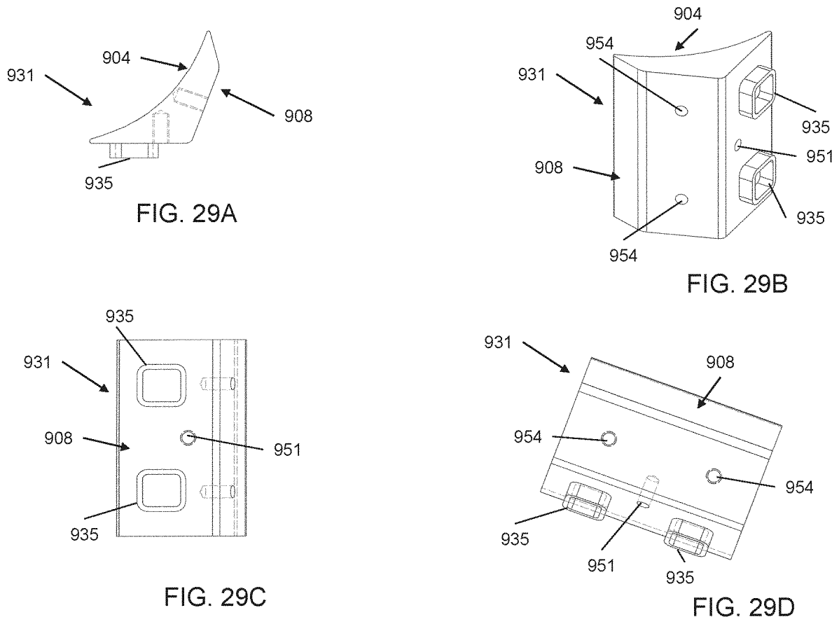

With reference to FIGS. 29A-29D, various views of wedge 931 are depicted. Wedge 931 has a limb facing side 904 and a socket attachment side 908 facing socket attachment 900, with the wedge having a generally triangular shape. In other embodiments the shape may any type of quadrilateral shape. The limb facing side 904 is depicted as being concave but in other embodiments may be, for example, flat or contoured to accommodate a limb or padding. The socket attachment side 908 may be angled (e.g., two sides of a triangle) and configured to conform to the final desired position of socket attachment members (e.g., first member 901 and middle plate 909). Socket attachment side 908 may have a pair of square protrusions 935, extending from the surface and configured (e.g., shaped and dimensioned) for insertion into holes 936 of middle plate 909 (see FIGS. 28B and 28D). Protrusions 935 are shaped and dimensioned to fit into holes 936 and thus in other embodiments, will vary in shape, dimensions, and quantity with the shape, dimensions, and quantity of holes 936. A fastener hole 951 extends into the socket attachment side 908 of wedge 931. Fastener hole 951 is alignable with fastener hole 938 of middle plate 909 when protrusions 935 are placed into holes 936 and fastener hole 951 is configured (e.g., shaped and dimensioned) to accommodate a fastener placed through fastener hole 938 and into fastener hole 951. Fastener holes 954 extends into the socket attachment side 908 of wedge 931. Fastener holes 954 are alignable with fastener holes 939 of member 901 and are configured (e.g., shaped and dimensioned) to accommodate fasteners placed through fastener holes 939 and into fastener holes 954.

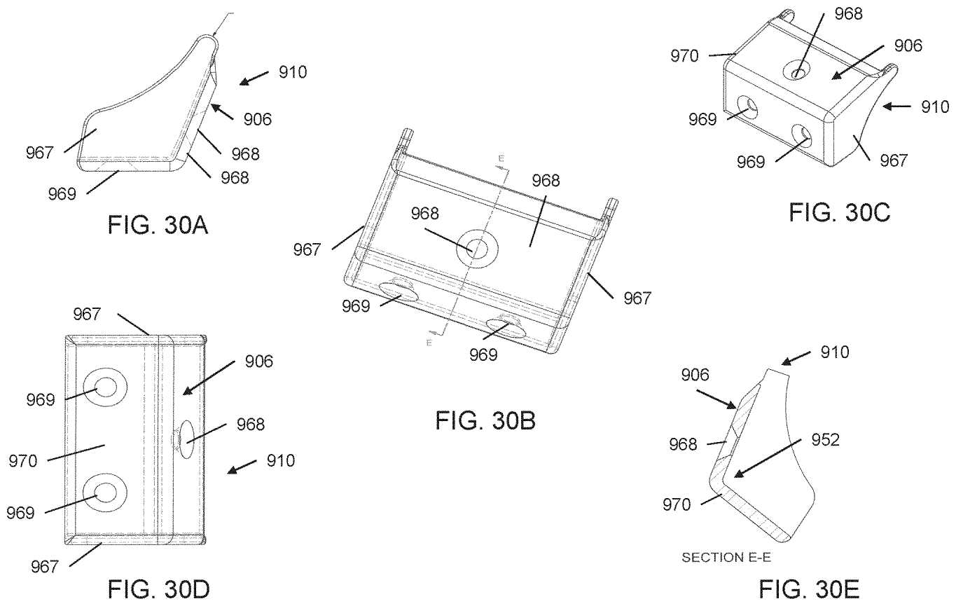

With reference to FIGS. 30A-30E, various views of cap 910 are shown. Cap 910 is depicted as a sleeve or cover for a portion of the outer surface 906 of member 901 and the outer surface 906 of at least a portion of middle plate 909. Cap 910 includes as an angled member 970 with an inner surface 952 configured (e.g., shaped and dimensioned) to make contact with part of the outer surface 906 of member 901 and the outer surface 906 of at least part of middle plate 909. Cap 910 has side panels 967 which cover the sides of plate 907 and at least part of the sides of middle plate 909. Cap 910 has a fastener hole 968 which aligns with fastener hole 938 and is configured to accommodate (e.g., shaped and dimensioned) a fastener, insertable through fastener hole 968, fastener hole 938, and into fastener hole 951. Cap 910 has fastener holes 969 which align with the fastener holes 939, and are configured to accommodate (e.g., shaped and dimensioned) a fastener, insertable through fastener holes 969, fastener holes 939, and into fastener holes 954.

With reference to FIGS. 29A-30E, certain embodiments of wedge 931 and cap 910 may be formed of materials such as, for example, aluminum, polypropylene, polyethylene, carbon fiber and epoxy resin, stainless steel, or combinations thereof.

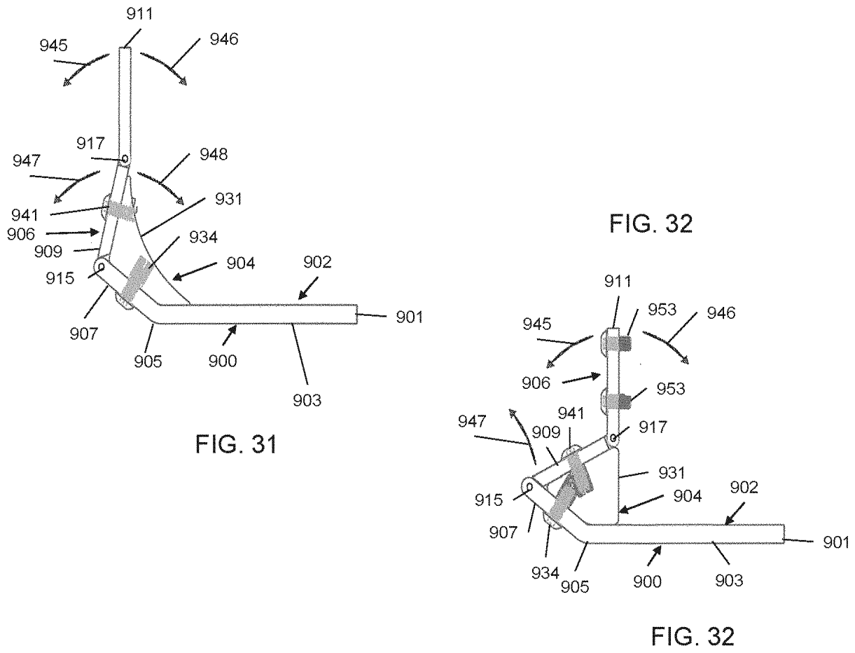

With reference to FIGS. 31 and 32, socket attachment 900 is shown in two configurations and with two different embodiments of wedge 931. Thus, the articulated segments (e.g., member 901, middle plate 909, and upper plate 911) socket attachment 900 may be adjusted to a desired position. Wedge 931 is configured (e.g., shaped and dimensioned) to maintain the desired position when socket attachment facing side 908 is placed in contact with member 901 and plate 909, with protrusions 935 placed into holes 936 and a fastener 941 is placed through fastener hole 938 and into fastener hole 951, and fasteners 934 are placed through fastener holes 939 and into fastener holes 954. While two possible configurations are depicted, one skilled in the art would understand the various other possible configurations of socket attachment 900 and wedge 931.

As depicted upper plate 911 may be pivoted on hinge 917 with respect to middle plate 909 in the direction of arrows 945 or 946. Middle plate 909 may be pivoted on hinge 915 with respect to its orientation to member 901 in the direction of arrows 947 or 948.

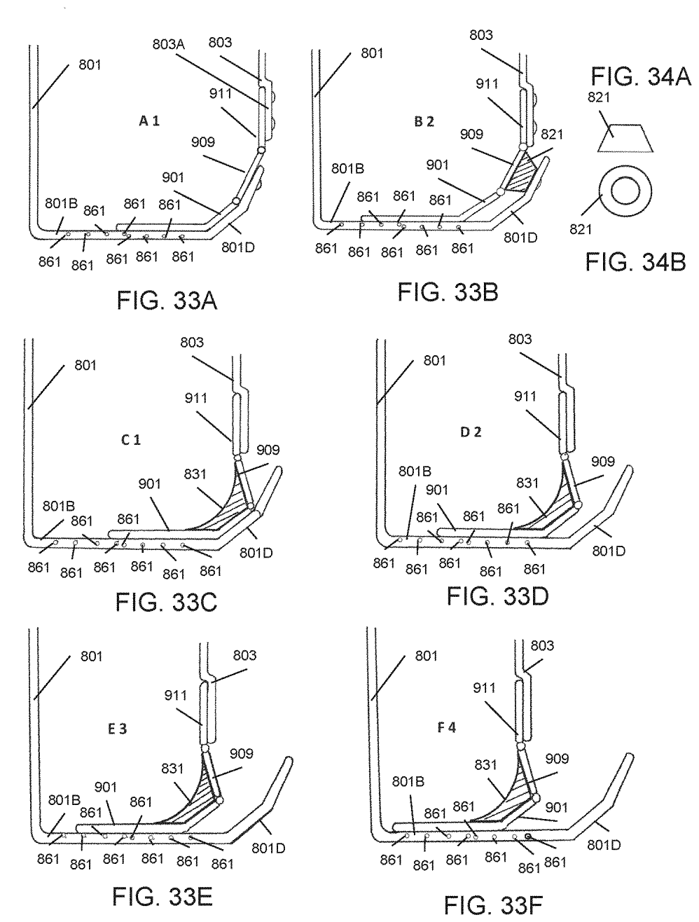

With reference to FIGS. 33A-33F, various cross-sectional side views of examples of configurations of socket attachment 900 connected to front shell 801 and rear shell 803 of a socket (e.g., socket 800) are depicted. Front shell 801 is shown with positioning holes 861 through base 801B from the limb facing side of base 801B to the pylon facing side of 801B, and distributed between the front shell surface (e.g., front shell surface 801A) of front shell 801 and the rim 801D. Rim 801D may be, for example, straight or segmented, with the segments at angles to each other. Member 901 may be connected to base 801B and upper plate 911 may be connected to rear shell 803, with middle plate 909 hingedly connected to member 901 at the first end and upper plate 911 at the second end.

With reference to FIG. 33A, member 901 may be attached to base 801B and in surface contact with rim 801D (e.g., plate 907 may be in contact with rim 801D. A part of middle plate 909 may also be, for example, in contact with the surface of rim 801D or attached to rim 801D by a fastener. FIG. 33A depicts an example of a configuration for sizing parameter A1.

With reference to FIG. 33B, member 901 may be attached to base 801B. A spacing wedge 821 may be inserted between rim 801D and middle plate 909, to maintain a desired position of the socket attachment 900. Wedge 821 may be attached to rim 801D and middle plate 909 by a fastener. Member 901 may be attached at a position where member 901 is closer to the front shall (e.g., front shell surface 801A), than as depicted in FIG. 33A. There may be a space between a section of member 901 (e.g., plate 907) and rim 801D. FIG. 33B depicts an example of a configuration for sizing parameter B2.

With reference to FIGS. 34A-B, an embodiment of wedge 821 is depicted from a side view and a top view, respectively. Wedge 821 is depicted here as having a cylindrical trapezoid shape, with a base wider than its top. However, with respect to FIGS. 33B, and 34A-B, wedge 821 may be, for example, any shape (e.g., see FIG. 24) suitable for positioning and supporting middle plate 909 by placement between rim 801D and middle plate 909. Wedge 821 may be, for example, another embodiment of wedge 821A, 821B, or 821C.

With reference to FIG. 33C, member 901 may be attached to base 801B. Wedge 831 may be inserted and connected to base 901 and middle plate 909, to maintain a desired position of the socket attachment 900. There may be a space between middle plate 909 and rim 801D. FIG. 33C depicts an example of a configuration for sizing parameter Cl.

With reference to FIG. 33D, member 901 may be attached to base 801B. Wedge 831 may be inserted and connected to base 901 and middle plate 909, to maintain a desired position of the socket attachment 900. Member 901 may be attached at a position where member 901 is closer to the front shall (e.g., front shell surface 801A), than as depicted in FIG. 33A. There may be a space between a section of member 901 (e.g., plate 907) and rim 801D and there may be a space between middle plate 909 and rim 801D. FIG. 33D depicts an example of a configuration for sizing parameter D2.

With reference to FIG. 33E, member 901 may be attached to base 801B. Wedge 831 may be inserted and connected to base 901 and middle plate 909, to maintain a desired position of the socket attachment 900. Member 901 may be attached at a position where member 901 is closer to the front shall (e.g., front shell surface 801A), than as depicted in FIG. 33D. There may be a space between a section of member 901 (e.g., plate 907) and rim 801D and there may be a space between middle plate 909 and rim 801D. FIG. 33D depicts an example of a configuration for sizing parameter E3.

With reference to FIG. 33F, member 901 may be attached to base 801B. Wedge 831 may be inserted and connected to base 901 and middle plate 909, to maintain a desired position of the socket attachment 900. Member 901 may be attached at a position where member 901 is closer to the front shall (e.g., front shell surface 801A), than as depicted in FIG. 33E. There may be a space between a section of member 901 (e.g., plate 907) and rim 801D and there may be a space between middle plate 909 and rim 801D. FIG. 33D depicts an example of a configuration for sizing parameter F4.

With reference to FIGS. 33A-33F, sizing configurations are identified by parameters A1, B2, Cl, D2, E3, and F4. An anterior/posterior length may be the length between the front shell surface (e.g., front shell surface 801A) and the rear shell 803. Sizing parameter A1 may represent, for example, an anterior/posterior length of approximately 6.05″. Sizing parameter B2 may represent, for example, an anterior/posterior length of approximately 5.5″. Sizing parameter Cl may represent, for example, an anterior/posterior length of approximately 5.0″. Sizing parameter D2 may represent, for example, an anterior/posterior length of approximately 4.5″. Sizing parameter E3 may represent, for example, an anterior/posterior length of approximately 4.0″. Sizing parameter F4 may represent, for example, an anterior/posterior length of approximately 3.5″. Spacing wedge 821 and wedge 931, may be used for positioning socket attachment 900 but may also be used to aid in size reduction for the socket 800.

With reference to FIGS. 33A-33F, sizing configurations identified by parameters A1, B2, Cl, D2, E3, and F4 are examples of sizing configurations. However, other configurations may exist in other embodiments and may be used to achieve a desired fit for a limb. Furthermore, a plurality of wedge 931 or a plurality of positioning wedge 821 may be used to achieve a desired fit. There may also be embodiments where wedge 931 and positioning wedge 821 are used together to achieve a desired fit. In some embodiments, joint stabilizer cap 910 (see FIGS. 25C and 30A-30E) may be connected to plate 907 and middle plate 909 (see FIGS. 25A25C), between socket attachment 900 and rim 801D. In some embodiments, joint stabilizer cap 910 (see FIGS. 25C and 30A-30E) may be connected to plate 907 (see FIGS. 25A-25C) and middle plate 909, between socket attachment 900 and rim 801D and may be used in conjunction with wedge 931 to find a suitable sizing fit for an amputee's limb.

With reference to FIGS. 30A-30E, the cap 910 may be configured (e.g., shaped and dimensioned) for placement on the outer surface 906 of second plate 907 and the outer surface 906 of middle plate 909 (e.g., see FIGS. 31 and 32). Cap 910 may be used, for example, to provide additional support to maintain the desired position of socket attachment 900 and/or as a cap to protect parts of the base (e.g., base 801B) or the rim (e.g., rim 801D) from direct contact with parts of the socket attachment 900 which may flex due to high stresses mobility loads.

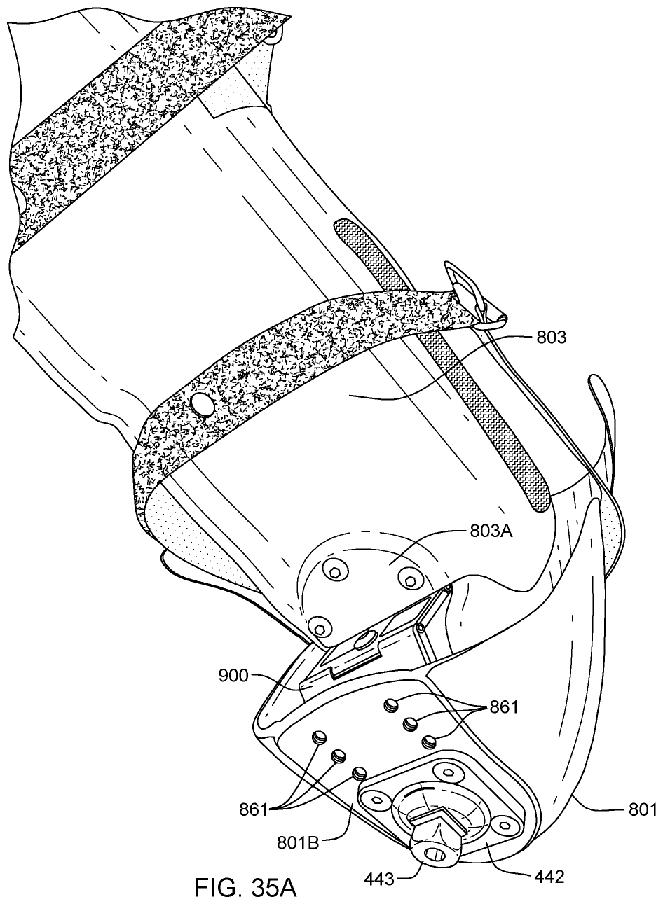

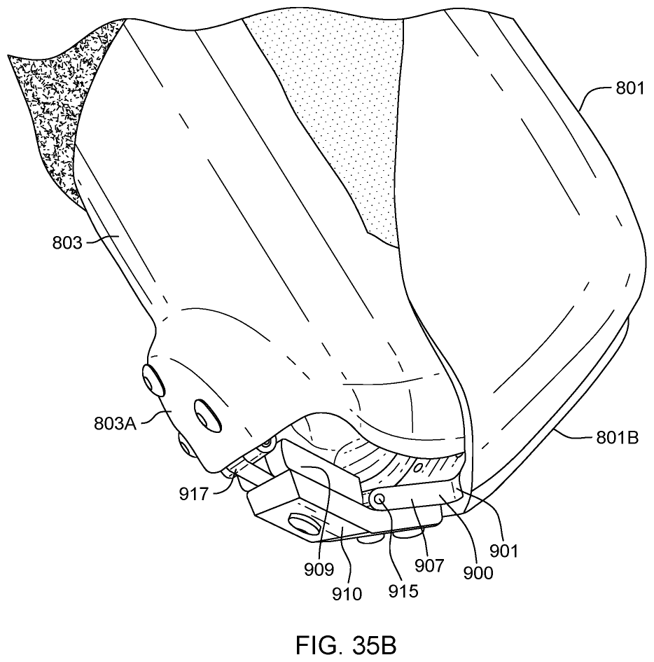

FIG. 35A is a bottom perspective view of the of the articulated socket plate system of FIG. 25A within the post-operative perpetual dimension prosthetic system 800 of FIG. 20, and with the rim 801D (see FIGS. 20, 21, and 33A-33F) removed to provide a clearer view of the connection between the socket attachment 900 and the front shell 801 and the rear shell 803, forming the articulated socket system. With continued reference to FIG. 35A, rear shell 803 is depicted as open or rotated about hinge 917 (not marked). With continued reference to FIG. 35A, base 801B of front shell 801 is shown with the plurality of pairs of positioning holes 861. The space between adjacent pairs of positioning holes may be, for example, be the same. In a preferred embodiment, seven pairs of holes may be provided. However, in other embodiments, there may be more or less than seven pairs.

With reference to FIGS. 25C-26D, fastener holes 921 on member 901 provide means for attaching socket attachment 900 to a base (e.g., base 801B of FIG. 35A) by fasteners, as will be described below. While four fastening holes 921 are depicted, there may be embodiments with less than four fastener holes 921 or embodiments with more than four fastener holes 921. Also, while fastener holes 921 are depicted as a means for accepting screws or bolts for attaching of member 901, there may be embodiments where other fasteners such as, for example, rivets, adhesives, epoxies, and/or similarly available fasteners may be used.

Referring to FIG. 35A, and embodiment of receiving plate 442 may be attached to base 801B and to member 901 of socket attachment 900. Receiving plate 441 may be connected to base 801B and member 901 by fasteners inserted through receiving plate 442, through a plurality of pairs of positioning holes 861, and through fastener holes 921. An embodiment of hardware connecting device 443 extends from receiving plate 442 and away from base 801B. As shown in FIG. 20, shaft 441 and artificial foot 445 may be connected to connecting device 443.

FIG. 35B is a bottom perspective view of the of the articulated socket plate system of FIG. 25A within a single socket embodiment prosthetic system of FIG. 20, and with cap 910 connected to the socket attachment 900. Rim 801D (see FIGS. 20, 21, and 33A-33F) has been removed to provide a clearer view of the connection between the socket attachment 900, the front shell 801 and the rear shell 803, forming the articulated socket system. Cap 910 is shown attached to member 901 at plate 907 by two fasteners and attached to middle plate 909 by a fastener.

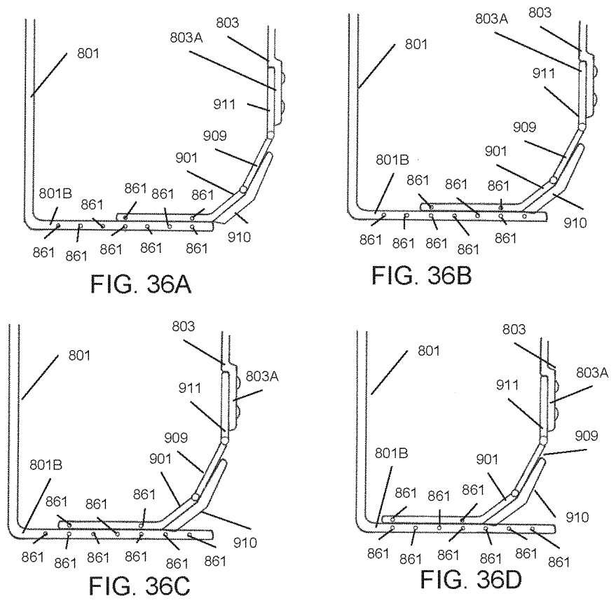

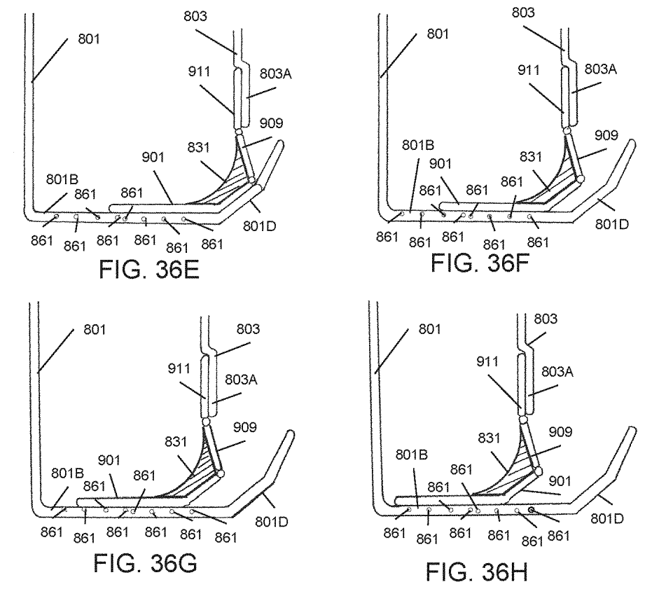

With reference to FIGS. 27A, 27C, 32, and 33A-33F (see also FIGS. 36A-36H described further below), fastener holes 919 on plate 911 provide means for attaching back shell 803 to socket attachment 900 by screws or bolts, as will be described below. While three fastener holes 919 are depicted, there may be embodiments with less than three fastener holes 919 or embodiments with more than three fastener holes 919. Also, while fastener holes are depicted as a means for accepting screws or bolts for attachment to socket attachment 900, there may be embodiments where other fastening means such as, for example, rivets, adhesives, or epoxies may be used.

The post-operative perpetual dimension prosthetic system includes a single socket (e.g., single socket 800 of FIG. 20-21) connected to socket attachment 900. Thus, the single socket includes configurations (e.g., sizes and dimensions) that provide for post-operation use and for post-recovery use as a prosthetic device. With reference to FIG. 18, front shell 801 has an extended surface 801A that is shaped to fit against the front of a trans-tibial amputee's leg or limb. Extended surface 801A of the front shell terminates in a base 801B that is configured (e.g., shaped and dimensioned) to be below the bottom of the amputee's limb. Dome 831 provides room to accommodate the amputee's knee. As discussed below, front shell 801 is configured (e.g., sized and dimensioned) to be the main weight bearing part of the single socket system and may be formed of a rigid load bearing material. The preferred embodiment of a material for the front shell 801 may be, for example, carbon fiber and epoxy resin. The carbon fiber and epoxy resin combination is easy to work with and form into a desired shape and is strong enough to bear the full load of an amputee's body when walking or running. Alternate embodiments may use rigid materials, such as, for example, aluminum, polypropylene, polyethylene, carbon fiber and epoxy resin, stainless steel, or combinations thereof.

With reference to FIG. 20 socket 800 is the interface socket that an amputees' leg may be inserted into shortly after undergoing a trans-tibial amputation. FIG. 18 shows the front shell 801, with baseplate 801B configured (e.g., shaped and dimensioned) to fit under the limb and the extended shell 801A configured (e.g., shaped and dimensioned) to fit along the anterior side of the limb. As may be seen in FIG. 19, rear shell 803 has an extended surface 805 configured (e.g., shaped and dimensioned) to fit against the rear of the leg of an amputee. Rear shell 803 may be made of a pliable and formable material. In the preferred embodiment it may be made of, for example, polyethylene but alternate embodiments may use similar materials, including polypropylene. Rear shell 803 includes the upper rear shell 805 with left side wing and right side wing. (References to right and left herein are from the perspective of the amputee that would be using the prosthetic device, such as the amputee would be facing in the same direction as the front of the prosthetic apparatus faces).

In certain embodiments the front shell 801 and rear shell 803 of FIGS. 18 and 19, may be attached by and to articulated double hinged socket attachment 900 of FIGS. 25A-D. Referring to FIG. 20 single socket embodiment 800 includes front shell 801 connected to the rear shell 803 by hinge 711 shown in outline since it is inside the shells. In place of articulated hinge 711, socket attachment 900 of FIGS. 25A-D may be used in certain embodiments as shown in FIGS. 33A-33E, 35A, and 35B (see also FIGS. 36A-36H described further below). Plate 901 may attach to a top inside or limb facing surface of base 801B and upper plate 911 may attach to the lower portion of rear shell 803A. Middle plate 909 of socket attachment 900 may connect at pivot points 917 and 915 (e.g., hinge 917 and hinge 915) with upper plate 911 and member 901, respectively, and thus may move when member 901 or upper plate 911 move. Rear shell 803 may be connected to upper plate 911, similar to how upper plate 711C is connected to rear shell 803 at 803A, as shown in FIGS. 19 and 24.

As shown in FIGS. 25A-D, for socket attachment 900 a single wedge may be configured (e.g., sized and dimensioned) to achieve desired spacing and sizing. Plate 711C is shown in FIG. 24 connected to a lower portion 803A of rear shell 803, and similarly, upper plate 911 of FIGS. 25A-D may be connected to a lower portion 803A of rear shell 803. With reference to FIG. 24, plate 711A of hinge 711 connects to the inside of bottom base 801B of the front shell Rear rim 801 and similarly with reference to FIGS. 25A-D, member 901 of socket attachment 900 may connect to the inside of the bottom of base 801B of the front shell 801. With reference to FIGS. 31 and 32, positioning wedge 931 may be positioned between middle plate 909 of socket attachment 900, with the positioning wedge 931 positioned on the limb facing side 902 of socket attachment 900. Wedge 931 is depicted as connected to middle plate 909 by a fastener 941 inserted through middle plate 909, towards a user's limb, and a plurality of fasteners 933 extending through second plate 907 of member 901, towards a user's limb. Fastener 941 and each of fasteners 933 may be, for example, screws, bolts, nails, or similar fasteners, or combinations thereof. Furthermore, fastener 941 is depicted as a single fastener extending through middle plate 909, but more than one fastener of the type of fastener 941 may be used. Fasteners 933 extending through member 901 is depicted as two fasteners, but one fastener or more than two fasteners of the type of fasteners 933 may be used.

With continued reference to FIGS. 25A-D, 31, and 32, middle plate 909 of socket attachment 900 may connect at pivot points 917 and 915 with upper plate 911 and member 901, respectively, and thus may move when member 901 or upper plate 911 move. With the presence of wedge 931 connected to middle plate 909 and member 901, movement of middle plate 909 and member 901 may be fixed into position relative to each other, once user sizing is complete. The wedge may be, for example, sized to maintain the desired position of the plates and members of socket attachment 900 and front shell 801 and rear shell 803. Once the wedge 931 is sized, positioned, and connected, the wedge aids in maintaining the position of socket attachment 900 and the overall fit and comfort of the front shell 801 and rear shell 803 around the user's limb.

With reference to FIGS. 21-23, the artificial foot 445 is connected by shaft 441 to hardware 443 and a reinforced bottom connection plate 442 and base 801B of front shell 801. Reinforced connection plate 442 is a reinforced portion of base 801B of front shell 801 configured (sized and dimensioned) and formed of a material for carrying the load put on it by the amputee when walking or standing. This reinforced portion may be made from, for example, aluminum, carbon fiber and epoxy resin, stainless steel, or similar metals, plastics, composites, or combinations thereof.

With reference to FIGS. 21-23, 35A, and 25A-26C, socket attachment 900 is shown connected to the front shell 801 and the rear shell 803 at the lower portion of rear shell 803A. As shown, connection plate 442 is connected to hardware 443, and connection plate 442 is shown connected to base 801B by 4 bolts, inserted through positioning holes in the connection plate 801B and through two of the plurality of pairs (e.g., the plurality of pairs 861). The connection plate 801B is depicted as substantially square, but other shapes may be suitable. The positioning holes 861 in the connection plate 801B are positioned towards the sides and are positioned to align with the two pairs of fastener holes (e.g., fastener holes 921), with one pair of fastener holes 921 fastened to base 801B closer to front shell 801 and a second pair of holes closer to the rear shell 803. In certain embodiments, the two of the plurality of pairs of fastener holes 921 may be aligned with adjacent positioning holes 861 or may be separated to span, for example, 4 pairs of positioning holes 861 (i.e., two pairs of positioning holes 861 between positioning holes 861 aligned with fastener holes 921). With reference to FIGS. 35B, there are seven pairs of positioning holes 861. Three pairs of positioning holes 861 are visible and four pairs of positioning holes are covered by connection plate 442, with two pairs aligned with holes in the connection plate through which bolts are inserted, and two pairs being in between the two pairs with bolts.

FIG. 21 shows how the space between the front shell 801 and the rear shell 803 may be varied by articulated socket attachment 900 as shown in FIGS. 33A-33F. As noted above, since plate 909 of socket attachment 900 is hingedly connected to plate 911 and member 901, socket attachment 900 may be moved and positioned such that rear shell 803 may be moved with respect to front shell 801 and the space between the two shells may be adjusted. After an amputation of a limb, the remaining portion of the amputated limb varies in size over the course of the amputee's recovery from the surgery. Initially the limb will swell as a result of the trauma of the surgery. Subsequently the limb will also shrink as the patient recovers, because there may be a need to minimize use of the limb during recovery. With a post-operative transitional amputation the knee of the amputee may be immobilized until the surgical wound created by the surgery has fully healed.

With reference to FIGS. 20, 21, 33A-33F, 35A, and 35B sizing configurations are identified by parameters A1, B2, Cl, D2, E3, and F4. An anterior/posterior length may be the length between the front shell surface (e.g., front shell surface 801A) and the rear shell 803. A medial/lateral length may be the length measured in relation to the medial/lateral sides of a limb inserted into the socket (e.g., socket 800). A circumference may be the space formed by the limb facing surface of front shell 801 and the limb facing surface of rear shell 803 configured to accommodate a limb inserted in the socket (e.g., socket 800).

The following direction terms refer to relative positions in relation to a part of a limb. The term “distal” refers to a direction towards the part of the limb furthest from the knee; the term “proximal” refers to a direction towards the part of the limb closest to the knee; the term “anterior” refers to a direction towards the front of the limb; the term “posterior” refers to a direction towards the rear of the limb; the term “medial” refers to a direction the interior facing part of the limb and towards a midline of the body; and the term “lateral” refers to the exterior facing part of the limb and away from the midline of the body. The following measurements may, for example, be used after confirming whether the socket left leg or right leg is to receive the socket. The length of a patient's residuum patellar tendon to the distal end of the limb and the anterior to posterior measurement of the distal end of the limb, including bandages, liners, and/or socks (bandages, liners, and/or socks sizes may be estimated if not yet applied). The limb is intended to be placed within the socket, and the socket is preferred to have an anterior/posterior length within the space to accommodate the distal end of the limb that is larger than the anterior/posterior length of the distal end of the limb with bandages, liners, and/or socks. Additional measurements may include: the proximal anterior to posterior length measured at (and perpendicular to) the tibial tubercle; the distal medial to lateral length, measured at the widest distal end of the limb; the proximal medial to lateral length measured at the widest portion of the knee; the distal circumference, measured 3″ to 5″ above the distal end; and the proximal circumference, measured at and perpendicular to the tibial tubercle. The distal anterior/posterior measurement may be a fixed socket setting (and requires specialized setting after establishing the initial setting). All other measurements may be adjusted as the limb heals.

Sizing parameters for socket 800 and socket attachment 900 are generally slightly larger than actual limb measurements. Sizing parameter A1 may represent, for example, an anterior/posterior length of approximately 6.05″, a medial/lateral length of approximately 6.0″, and a circumference of approximately 17.5″ to approximately 19.0″. Sizing parameter B2 may represent, for example, an anterior/posterior length of approximately 5.5″, a medial/lateral length of approximately 5.5″, and a circumference of approximately 16″ to approximately 17.25″. Sizing parameter Cl may represent, for example, an anterior/posterior length of approximately 5.0″, a medial/lateral length of approximately 5.0″, and a circumference of approximately 14.75″ to approximately 15.75″. Sizing parameter D2 may represent, for example, an anterior/posterior length of approximately 4.5″, a medial/lateral length of approximately 4.5″, and a circumference of approximately 12.75″ to approximately 14.5″. Sizing parameter E3 may represent, for example, an anterior/posterior length of approximately 4.0″, a medial/lateral length of approximately 4.0″, and a circumference of approximately 11.25″ to approximately 12.5″. Sizing parameter F4 may represent, for example, an anterior/posterior length of approximately 3.5″, a medial/lateral length of approximately 3.5″, and a circumference of approximately 10″ to approximately 11″. Spacing wedge 821 and wedge 931, may be used for positioning socket attachment 900 but may also be used to aid in size reduction for the socket 800.

Other embodiments of socket 800 may be made in four configurations (a first socket and a second socket for the right leg and a first socket and a second socket for the left leg) that fit approximately 95% of all trans-tibial amputees. Such configurations may include limb circumferences from about 10″ to about 20″. Sizing parameters for socket 800 and socket attachment 900 are generally slightly larger than actual limb measurements. A first socket configuration may have a socket length of 7″ and a second socket configuration may have a socket length of 9″. The socket length refers to the socket (e.g., socket 800) configured (e.g., shaped and dimensioned) to accept a maximum residual limb length of an amputee's leg (the “residuum”) for which a prosthesis is required. A patient's residuum length is measured from the mid patellar tendon (also referred to as the mid patellar ligament) is attached, to the distal portion of the amputated stump. For a residuum of more than 7″ to about 9″ in length, a 9″ socket configuration may be used by such patient. For a residuum of more than 5″ to about 7″ in length, a 7″ socket configuration may be used by such patient.

After patient measurements have been taken and with reference to FIGS. 36A-36H, the socket attachment may be positioned and connected to the base 810B by first plate 903 of member 901. Upper plate 911 may be connected to rear shell 803. With reference to FIGS. 36A-36D joint stabilizer 910 may be connected to socket attachment 900, more particularly to second plate 907 and middle plate 909. Rim 801D (see FIGS. 20, 21, 33A-33F, and 36E-36H) is not depicted but may be present in certain other embodiments where a joint stabilizer 910 may be connected to the socket attachment 900. The positioning of first plate 903 on base 810B in the anterior/posterior direction (e.g., with the front shell 801A being the anterior and rear shell 803 being the posterior) to accommodate a residuum may provide for anterior posterior length sizes of approximately 5″ to approximately 6.5″, with preferred settings at approximately 5.00″ (e.g., FIG. 36A), approximately 5.50″ (e.g., FIG. 36B), approximately 6.00″ (e.g., FIG. 36C), and approximately 6.50″ (e.g., FIG. 36D). These lengths may, for example, accommodate a distal end of a limb that is slightly smaller than the anterior/posterior length size setting. After the socket attachment 900 is connected to the base 810B, the anterior posterior length may further be adjusted to accommodate smaller limbs or to refine and adjust sizing for comfort. The joint stabilizer 910 may be removed and wedge 931 connected to adjust the size. Furthermore, rim 810D is depicted in FIGS. 36E-36H, however rim 801D may be removed in certain other embodiments. Using wedge 931, the anterior/posterior length may be adjusted from approximately 3.8″ to approximately 5.3″, with preferred settings at approximately 3.80″ (e.g., FIG. 36H), approximately 4.30″ (e.g., FIG. 36G), approximately 4.80″ (e.g., FIG. 36F), and approximately 5.30″ (e.g., FIG. 36E). These lengths may, for example, accommodate a distal end of a limb that is slightly smaller than the anterior/posterior length size setting. Thus, sizing of the socket 800 with socket attachment 900 may provide for socket sizes that range from a distal anterior/posterior size of approximately 3.80″, a proximal anterior/posterior size of approximately 3.60″, a distal medial/lateral size of approximately 3.85″, a proximal medial/lateral size of approximately 4.30″, a distal circumference of approximately 11.00″, and a proximal circumference of approximately 12.00″ to socket sizes of a distal anterior/posterior size of approximately 6.50″, a proximal anterior/posterior size of approximately 6.25″, a distal medial/lateral size of approximately 6.00″, a proximal medial/lateral size of approximately 7.00″, a distal circumference of approximately 19.00″, and a proximal circumference of approximately 20.5″.

In other embodiments, the wedge 931 may be configured (e.g., shaped and dimensioned) to provide for other size settings in between approximately 3.8″ and approximately 5.3″. In other embodiments, wedge 931 may be configured (e.g., shaped and dimensioned to provide for size settings less than 3.8″. Furthermore, in other embodiments the cap 910 may be configured (e.g., shaped and dimensioned) to provide for other size settings between approximately 5″ and approximately 6.5″. In still other embodiments, cap 910 may be configured (e.g., shaped and dimensioned) and wedge 931 may be configured (e.g., shaped and dimensioned) to be used in combination to provide for sizing between approximately 3.8″ and approximately 6.5″. In other embodiments, the socket 800 and socket attachment 900 may be configured (e.g., shaped and dimensioned) to provide for sizing greater than 6.5″ by providing additional positioning holes 861 in base 801B or adjusting spacing of the positioning holes on base 801B.

While several aspects of the present invention have been described and depicted herein, alternative aspects may be affected by those skilled in the art to accomplish the same objectives. Accordingly, it is intended by the appended claims to cover all such alternative aspects as fall within the true spirit and scope of the invention.

What is claimed is:

1. A single socket adjustable prosthetic system comprising:
a. a first shell with an extended surface configured to fit against a first side of an amputee's limb, the surface terminating at a base, the base configured to extend under the amputee's limb and the base having a limb facing surface and a bottom surface;
b. a second shell with an extended surface configured to fit against a second side of the amputee's limb;
c. an articulated hinge having a first member, a middle member, and a second member, the first member comprising a first plate connected to a second plate at an angle to each other, with the second plate hingedly connected to a first end of the middle member and the second member hingedly attached at a second end of the middle member, with the first plate removably connected to the base and the second member of the articulated hinge attached to the second shell;
d. the first plate further having fastening holes configured to align with positioning holes in the base to adjust the relative anterior/posterior position of the first plate and the base prior to connection, the fastening holes and positioning holes configured to accommodate removable fasteners; and
e. the articulated hinge connected at the first member to the base of the first shell and connected at the second member to the second shell forms a space between the first shell and the second shell, the space to be varied to hold, and accommodate size, swelling, and shrinking of the amputee's limb.

2. The single socket adjustable prosthetic system of claim 1, further comprising:
a hinge positioning and securing system; wherein
the base of the first shell has a rim around a rear portion of the base opposite the connection of the extended surfaces connection to the base;
the hinged attachment of the first member to the middle member is positioned at the base of the rim when the first member is connected to the interior of the base; and
the space between the middle member and the rim is adjusted by a wedge connected to the middle member and the first member.

3. The single socket adjustable prosthetic system of claim 1 further comprising a hinge positioning and securing system wherein:
the base of the first shell has a rim around a rear portion of the base opposite the connection of the extended surfaces connection to the base;
the hinged attachment of the first member to the middle member is positioned at the base of the rim when the first member is connected to the interior of the base; and
the space between the middle member and the rim is adjusted by at least one wedge inserted between the middle plate and the rim.

4. The single socket adjustable prosthetic system of claim 1, further comprising:
a shaft connected at a top end to the bottom surface of the base and the shaft projecting from the base to a bottom end; and
a prosthetic foot attached to the bottom end of the shaft; and
wherein the single socket adjustable prosthetic system when attached to the limb of the amputee can be used to stand and for mobility.

5. The single socket adjustable prosthetic system of claim 1 wherein the first shell comprises a rigid, load bearing material and the second shell comprises a pliable and formable material.

6. The single socket adjustable prosthetic system of claim 5 wherein the first shell material is a carbon fiber and epoxy resin.

7. The single socket adjustable prosthetic system of claim 5 the second shell material is polyethylene.

8. The single socket adjustable prosthetic system of claim 1 wherein:
the amputee has had a trans-tibial amputation and the first side of the amputee's limb is the front of the limb and the second side of the amputee's limb is the rear of the limb;
the second shell has an upper portion such that when the single socket adjustable prosthetic system is attached to the amputee's limb the amputee is prevented from bending of the knee;
the first shell has an upper portion that covers and protects the knee; and
a trim line can be cut that removes portions of the upper portion of the second shell and can remove portions of the upper portion of the first shell, to allow the amputee to flex the knee when the single socket is attached to the amputee's leg.

9. The single socket adjustable prosthetic system of claim of claim 8, wherein the trim line cut of the upper portion of the second shell and the first shell is selected from the group consisting of: a supra condylar supra patellar (SCSP) trim line, a supra condylar (SC) trim line, and a patellar tendon bearing (PTB) trim line.

10. The single socket adjustable prosthetic system of claim 1, wherein the removable fasteners have threading.

11. The single socket adjustable prosthetic system of claim 2, wherein the fastening holes have threading to accommodate the removable fasteners.

12. The single socket adjustable prosthetic system of claim 1 wherein the first member of the articulated hinge is attached to the limb facing surface of the base and the second member of the articulated hinge is attached to a limb facing surface of the second shell.

13. The single socket adjustable prosthetic system of claim 1, wherein:
seven pairs of positioning holes are positioned between an anterior and a posterior end of the base, and each of the pairs comprise a first positioning hole positioned towards a medial side of the base and a second positioning hole positioned towards a lateral side of the base; and
two pairs of fastener holes are positioned between an anterior and a posterior end of the first plate, and each of the pairs comprise a first fastener hole positioned towards a medial side of the first plate and a second fastener hole positioned towards a lateral side of the base;
the two pairs of fastener holes configured for alignment with a first pair of positioning holes and a second pair of positioning holes.

14. The single socket adjustable prosthetic system of claim 1, wherein the articulated hinge further comprises a stabilizer cap connected to a base facing side of the second plate and the middle member.

15. The single socket adjustable prosthetic system of claim 1, wherein the space varied to hold and accommodate size, swelling and shrinking of the amputee's limb, is configured to accommodate a limb, the space having a circumference from about 10″ to about 20″.

16. The single socket adjustable prosthetic system of claim 1, further comprising a socket length configuration selected a group consisting of a 7″ socket length configuration to accept a left side residuum length of greater than 5″ to 7″, a 7″ socket length configuration to accept a right side residuum length of greater than 5″ to 7″, a 9″ socket length configuration to accept a left side residuum length of greater than 7″ to 9″, and a 9″ socket length configuration to accept a right side residuum length of greater than 7″ to 9″.

17. The single socket adjustable prosthetic system of claim 16, wherein the articulated hinge with the stabilizer cap is positioned on the baseplate and the anterior posterior length is from 5″ to 6.5″.

18. The single socket adjustable prosthetic system of claim 16, wherein the articulated hinge with the positioning wedge is positioned on the baseplate and the anterior posterior length is from 3.8″ to 5.3″.
